(19) United States
(12) Patent Application Publication
(10) Pub. No.: US 2025/0101112 A1
(43) Pub. Date: Mar. 27, 2025

(54) KILLER CELL LECTIN-LIKE RECEPTOR SUBFAMILY G MEMBER 1 (KLRG1) DEPLETING ANTIBODIES

(71) Applicant: ABCURO, INC., Newton, MA (US)

(72) Inventors: Stefano Vincenzo GULLA, Newton, MA (US); Kenneth Evan THOMPSON, Newton, MA (US)

(21) Appl. No.: 18/291,013

(22) PCT Filed: Jul. 26, 2022
(86) PCT No.: PCT/US2022/038310

§ 371 Date: Jan. 22, 2024

Related U.S. Application Data
(60) Provisional application No. 63/225,828, filed on Jul. 26, 2021.
(60) Provisional application No. 63/308,222, filed on Feb. 9, 2022.

(51) Int. Cl. C07K 16/28; A61K 39/00; A61P 35/00; A61P 37/06

(52) U.S. Cl. CPC C07K 16/2851; A61P 35/00; A61P 37/06; A61K 2039/505; C07K 2317/24; C07K 2317/33; C07K 2317/34; C07K 2317/41; C07K 2317/565; C07K 2317/94

(57) ABSTRACT

The invention relates to antibodies, or fragments thereof, that specifically bind to KLRG1, methods of depleting killer cell lectin-like receptor GI (KLRG1) expressing T cells and/or NK cells in a subject in need thereof of, methods of treating a disorder associated with excess KLRG1-expressing T cells in a subject in need thereof, methods of treating cancer in a subject, adjunct therapies for treatment of cancer in a subject, methods of depleting KLRG1-expressing cells in a mixed population of cells, methods of selectively depleting KLRG1-expressing CDS effector T cells, pharmaceutical compositions of anti-KLRG1 antibodies, and kits for anti-KLRG1 antibodies.

### CROSS-REFERENCE TO RELATED APPLICATIONS

This application claims the benefit of U.S. Provisional Patent Application 63/225,828, filed Jul. 26, 2021, and U.S. Provisional Patent Application 63/308,222, filed Feb. 9, 2022, the entire contents of which are incorporated herein by reference in their entireties.

### BACKGROUND OF THE INVENTION

Cytotoxic T cells or NK cells may result in disease either through inappropriate cellular injury (e.g., autoimmunity) or through uncontrolled proliferation (e.g., certain leukemias and lymphomas involving cytotoxic T cells or NK cells). Injury to tissues by cytotoxic T cells are implicated in autoimmune diseases, type 1 diabetes, solid organ transplant rejection, and graft versus host disease. Uncontrolled proliferation of cytotoxic T cells or NK cells are also implicated in certain T cell leukemias and lymphomas, such as hepatosplenic T cell lymphoma and NK/T cell lymphoma. Killer cell lectin-like receptor G1 (KLRG1), a cell surface marker known to be present on mature cytotoxic T cells, has been demonstrated to be present on cytotoxic T cells with high-killing potential (WO2018053264). Thus, a need exists for compositions and methods for depleting KLRG1-expressing T cells and/or NK cells in subjects in need thereof.

### SUMMARY OF THE INVENTION

Disclosed herein, among a number of different embodiments, are antibodies, and fragments thereof, that specifically bind to an extracellular domain of KLRG1, and methods of depleting KLRG1-expressing T cells and/or NK cells using such antibodies and fragments in a subject in need of depletion treatment.

In one aspect, the disclosure relates to antibodies, or fragments thereof, that specifically bind to an extracellular domain of KLRG1 comprising a heavy chain variable region comprising an amino acid sequence of SEQ ID NO:4, and a light chain variable region comprising three complementarity determining regions (CDRs) comprising amino acid sequences SEQ ID NO:11 (CDR-L1), SEQ ID NO:12 (CDR-L2), and SEQ ID NO:13 (CDR-L3). In some embodiments the antibody, or fragment thereof, comprises a light chain variable region comprising SEQ ID NO:5. In some embodiments, the antibody, or fragment thereof, comprises a heavy chain comprising SEQ ID NO:6. In some embodiments, the antibody, or fragment thereof, comprises a light chain comprising SEQ ID NO:7. In some embodiments, the antibody, or fragment thereof, comprises a heavy chain comprising SEQ ID NO:6 and a light chain comprising SEQ ID NO:7. In some embodiments, the antibody, or fragment thereof, specifically binds the epitope PLNFSRI (SEQ ID NO:14), or a fragment thereof comprising at least five contiguous amino acids. The antibody, or fragment thereof, may be a monoclonal antibody, or a fragment or derivative thereof. The antibody, or fragment thereof, may be a humanized antibody, or a fragment thereof. The KLRG1 may be human KLRG1 or cynomolgus KLRG1.

In some aspects the disclosure relates to methods of depleting KLRG1-expressing T cells and/or NK cells in a subject in need thereof comprises delivering to the subject a therapeutically effective amount of an antibody, or fragment thereof, disclosed herein, thereby depleting KLRG1-expressing T cells and/or NK cells in the subject.

Other aspects the disclosure relate to methods of treating a disorder associated with excess KLRG1-expressing T cells in a subject in need thereof, comprises delivering to the subject a therapeutically effective amount of an antibody, or fragment thereof, disclosed herein, thereby depleting KLRG1-expressing T cells. The disorder may be a transplant disorder. The disorder may be an autoimmune disease. The disorder may be inclusion-body myositis.

In one aspect the disclosure relates to methods of treating cancer in a subject, wherein the cancer comprises cancer cells that express KLRG1, comprising delivering to the subject a therapeutically effective amount of an antibody, or fragment thereof, disclosed herein, wherein the delivery to the subject depletes the cancer cells expressing KLRG1.

In another aspect the disclosure relates to the treatment of cancer in a subject with an adjunct therapy, wherein the subject is undergoing checkpoint therapy, (regardless of whether said cancer expresses KLRG1), wherein the adjunct therapy comprises delivering to the subject a therapeutically effective amount of an antibody, or fragment thereof, disclosed herein, wherein delivery depletes KLRG1-expressing pathogenic T cells and/or NK cells attacking self-tissues in the subject.

In one aspect the disclosure relates to a method of depleting KLRG1-expressing cells in a mixed population of cells, wherein the KLRG1-expressing cells comprise one or more cells selected from the group consisting of T cells, NK cells, cancer cells, and combinations thereof, comprising delivering to the mixed population of cells a therapeutically effective amount of an antibody, or fragment thereof, disclosed herein in an amount effective to deplete KLRG1-expressing T cells, NK cells, cancer cells, or combinations thereof, in the mixed population of cells.

Another aspect of the disclosure relates to a method of selectively depleting KLRG1-expressing CD8 effector T cells, but with relative sparing of naïve T cells and/or regulatory T cells, comprising delivering to a subject a therapeutically effective amount of an antibody, or fragment thereof, disclosed herein, thereby selectively depleting KLRG1-expressing CD8 effector T cells.

Other aspects of the disclosure relate to pharmaceutical compositions, comprising at least one antibody, or fragment thereof, disclosed herein and a pharmaceutically acceptable carrier.

The disclosure also relates to kits comprising at least one antibody, or fragment thereof, as disclosed herein and instructions for use.

### BRIEF DESCRIPTION OF THE DRAWINGS

FIG. 1 shows gene expression of human KLRG1 by hepatosplenic T cell lymphoma (HSTCL) neoplasms in comparison to normal spleen expression.

FIG. 2 shows gene expression of human KLRG1 by hepatosplenic T cell lymphoma (HSTCL) neoplasms in comparison to NK cell lines and G-D T cell lines.

FIG. 3 shows gene expression of human KLRG1 by hepatosplenic T cell lymphoma (HSTCL) neoplasms in comparison to peripheral T cell lymphoma (PTCL).

FIG. 4 shows gene expression of human KLRG1 by NK/T cell lymphoma (NKTCL) neoplasms.

FIG. 5 shows gene expression of human KLRG1 by mycosis fungoides neoplasms.

FIG. 6 shows gene expression of human KLRG1 by mycosis fungoides neoplasms in comparison to healthy cells.

FIG. 7 shows gene expression of human KLRG1 by T and NK cell lymphoma and leukemia cell lines, including KARPAS-384 (gamma-delta T cell line), KHYG-1 (aggressive NK cell leukemia), and MTA (aggressive NK cell leukemia).

FIG. 8 shows gene expression of human KLRG1 across T cell prolymphocytic leukemia (T-PLL) neoplasms.

FIG. 9 shows the impact of antibody ABC008 on KLRG1+CD8+ T cell populations in Cynomolgus monkey.

FIG. 10 shows the impact of antibody ABC008 on KLRG1+CD8+ T cell populations in three subjects suffering from inclusion body myositis (IBM).

FIG. 11 shows the impact of antibody ABC008 on CD3+CD57+ large granular lymphocyte (LGL) T cell populations in three subjects suffering from inclusion body myositis (IBM).

FIG. 12 shows the impact of various doses and regimens of antibody ABC008 on KLRG1+CD8+ T cell populations in Cynomolgus monkey.

FIG. 13 shows baseline CD8 KLRG1+/KLRG1− T cell proportions in nine subjects (three cohorts of three subjects) suffering from IBM.

FIG. 14 shows the depletion of CD8+KLRG1+ T cells in the three cohorts relative to the baseline values shown in FIG. 13.

FIG. 15 shows the impact of antibody ABC008 on regulatory T cells (Tregs) in three subjects suffering from IBM.

FIG. 16 shows the impact of antibody ABC008 on Tregs in nine subjects (three cohorts of three subjects) suffering from IBM. Comparative data for alemtuzumab is also shown.

FIG. 17 shows the impact of antibody ABC008 on central memory T cells in nine subjects (three cohorts of three subjects each) suffering from IBM. Comparative data for alemtuzumab is also shown.

FIG. 18 shows pharmacokinetic data for ABC008.

FIG. 19 shows pharmacokinetic data for ABC008 in nine subjects (three cohorts of three subjects each) who have received 0.1 mg/kg, 0.5 mg/kg, or 2.0 mg/kg of ABC008.

FIG. 20A shows sporadic Inclusion Body Myositis Physical Functioning Assessment (sIFA) scores over time for three subjects suffering from IBM who received ABC008.

FIG. 20B shows modified Timed Up and Go (mTUG) scores over time for three subjects suffering from IBM who received ABC008.

FIG. 20C summarizes changes in sIFA, Inclusion Body Myositis Functional Rating Scale (IBMFRS), mTUG, and Manual Muscle Testing (MMT12) scores after 56 days for three subjects suffering from IBM who received ABC008.

FIG. 21 shows in vitro potency data for ABC008, fucosylated ABC008 (ABC108), and isotype control against CD8+CD57+ LGLs.

FIG. 22 shows baseline CD8 KLRG1+/KLRG1− T cell proportions in eleven subjects suffering from IBM.

FIG. 23 shows the depletion of CD8+KLRG1+ T cells in the eleven subjects relative to the baseline values shown in FIG. 13.

FIG. 24 shows the impact of antibody ABC008 on CD3+CD57+ large granular lymphocyte (LGL) T cell populations in a first cohort of three subjects suffering from inclusion body myositis (IBM).

FIG. 25 shows the impact of antibody ABC008 on CD3+CD57+ large granular lymphocyte (LGL) T cell populations in a second cohort of three subjects suffering from inclusion body myositis (IBM).

FIG. 26 shows the impact of antibody ABC008 on CD3+CD57+ large granular lymphocyte (LGL) T cell populations in a third cohort of five subjects suffering from inclusion body myositis (IBM).

FIG. 27 shows the impact of antibody ABC008 on CD8 naïve T cells in three subject cohorts suffering from IBM. Comparative data for alemtuzumab is also shown.

FIG. 28 shows the impact of antibody ABC008 on CD8 central memory T cells in three subject cohorts suffering from IBM. Comparative data for alemtuzumab is also shown. Legend: circles, cohort 1; diamonds, cohort 2; inverted triangles, cohort 3; squares, alemtuzumab.

FIG. 29 shows the impact of antibody ABC008 on CD8 effector memory T cells (TEM) in three subject cohorts suffering from IBM. Comparative data for alemtuzumab is also shown. Legend: circles, cohort 1; diamonds, cohort 2; inverted triangles, cohort 3; squares, alemtuzumab.

FIG. 30 shows the impact of antibody ABC008 on CD8 terminally differentiated effector memory T cells (TEMRA) in three subject cohorts suffering from IBM. Comparative data for alemtuzumab is also shown.

FIG. 31A shows Inclusion Body Myositis Functional Rating Scale (IBMFRS) scores over time for multiple subjects suffering from IBM who received ABC008.

FIG. 31B shows Manual Muscle Testing (MMT) scores over time for multiple subjects suffering from IBM who received ABC008.

FIG. 31C shows modified Timed Up and Go (mTUG) scores over time for multiple subjects suffering from IBM who received ABC008.

### DETAILED DESCRIPTION

Killer cell lectin-like receptor GI (KLRG1) is a type II transmembrane protein which can function as a co-inhibitory receptor by modulating the activity of T and NK cells. The intracellular portion of KLRG1 contains an immunoreceptor tyrosine-based inhibitory motif (ITIM) domain responsible for co-inhibition of T cell receptor (TCR) mediated signaling. The extracellular portion of KLRG1 contains a C-type lectin domain whose known ligands are cadherins. KLRG1 ligands include E-cadherin, N-cadherin, R-cadherin, and combinations thereof.

The receptor KLRG1 is expressed on the cell surface of T and NK cells which bind to ligands on epithelial and mesenchymal cells. In humans, KLRG1 expression is generally confined to cells of the immune systems, and specifically to CD8 positive T cells, NK cells, and, to a lesser extent, CD4 positive T cells. KLRG1 expression has been associated with the late differentiated phenotype. As antigen-specific T cells differentiate they can acquire increased expression of cytotoxic molecules, and therefore can have increased cytotoxic potential.

Thus, KLRG1-expressing (or KLRG1+) T cells and/or NK cells can be pathogenic and are therefore an advantageous target for cell depletion therapy. For example, administering to a subject in need of an effective amount of KLRG1-depleting agent (e.g., a KLRG1-expressing cell-depleting agent) with antibody dependent cellular cytotoxicity (ADCC) effector function can eliminate or reduce the number of cytotoxic T cells and/or NK cells injuring healthy cells as, for example, in autoimmune diseases and in transplant disorders. Methods comprising administering a KLRG1-expressing cell-depleting agent, such as the antibodies and fragments thereof disclosed herein, are also advantageous in treating patients with cancer cells expressing KLRG1.

Depletion of KLRG1+ cells would be desirable in diseases with abnormal accumulations of KLRG1 or KLRG1+ cells in tissue samples. These include certain mature T and NK cell lymphomas and leukemias, in particular NK/T cell lymphoma (NKTCL), aggressive NK cell leukemia (ANKL), hepatosplenic T-cell lymphoma (HSTCL), gamma-delta T-cell lymphoma (GDTCL), NK/T cell lymphoma (NKTCL), aggressive NK cell leukemia (ANKL), T-cell prolymphocytic leukemia (T-PLL), adult T-cell leukemia/lymphoma (ATLL), angioimmunoblastic T-cell lymphoma (AITL), subacute panniculitis like T-cell lymphoma (SPTCL), enteropathy associated T-cell lymphoma (EATL), anaplastic large cell lymphoma (ALCL), cutaneous T-cell lymphoma (CTCL), peripheral T-cell lymphoma not otherwise specified (PTCL-NOS), T-cell lymphoma unclassified (TCL-UN), and T-cell large granular lymphocytic leukemia (T-LGLL). Additionally, for certain autoimmune diseases, it may also be desirable to deplete KLRG1+ cells, in particular inclusion body myositis (IBM), primary biliary cholangitis, primary sclerosing cholangitis, multiple sclerosis, rheumatoid arthritis, Crohn's disease, ulcerative colitis, oral lichen planus, vitiligo, Sjogren's syndrome, pure red cell aplasia, aplastic anemia, type 1 diabetes, lupus, lupus nephritis, alopecia areata, and Addison's disease.

Certain exemplary embodiments will now be described to provide an overall understanding of the principles of the structure, function, manufacture, and use of the antibodies, fragments thereof, compositions of matter, kits, and related methods and therapies disclosed herein. Those skilled in the art will understand that the disclosures described herein are exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

### Definitions

Unless otherwise defined, all technical and scientific terms used herein have the same meaning as commonly understood by one skilled in the art to which this present disclosure belongs. The terminology used in the description of the present disclosure herein is for the purpose of describing particular embodiments only and is not intended to be limiting of the present disclosure and any invention(s) described or otherwise provided for herein.

Amino acids are represented herein by either the one-letter code, or the three-letter code, both in accordance with established usage.

All publications, patent applications, patents, patent publications, and other references cited herein are incorporated by reference in their entireties for the teachings relevant to the sentence and/or paragraph in which the reference is presented.

As used in the description of the present disclosure and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as an amount of polypeptide, dose, time, temperature, enzymatic activity or other biological activity and the like, is meant to encompass variations of ±20%, ±10%, ±5%, 1%, +0.5%, or even ±0.1% of the specified amount.

The transitional phrase “consisting essentially of” means that the scope of a claim is to be interpreted to encompass the specified materials or steps recited in the claim, “and those that do not materially affect the basic and novel characteristic(s)” of the claimed invention.

The term “consists essentially of” (and grammatical variants), as applied to a polynucleotide or polypeptide sequence of this present disclosure, means a polynucleotide or polypeptide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence such that the function of polypeptide is not materially altered. The total of ten or less additional amino acids can include the total number of additional amino acids on both ends added together.

An “effective amount” as used herein is an amount that provides a desired effect. The term “effective amount” can refer to a dosage or amount of an antibody or antigen binding fragment that is sufficient to reduce the activity of KLRG1 to result in amelioration of symptoms in a patient or to achieve a desired biological outcome, e.g., reduced activity of KLRG1, modulation of lymphocyte co-inhibition response, increased or decreased activation of cytotoxic T and NK cells, or increased or decreased release of IFNγ by cytotoxic T cells or NK cells.

A “therapeutically effective amount” as used herein is an amount that provides some clinical improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

By the terms “treat,” “treating,” or “treatment of,” it is intended that the severity of the condition of the subject is reduced, or at least partially improved or modified, and that some alleviation, mitigation, or decrease in at least one clinical symptom is achieved.

The term “depletes” as used herein with respect to T cells and/or NK cells and/or KLRG1-expressing cancer cells refers to a measurable decrease in the number of said cells in a subject or in a sample. The reduction can be at least about 10%, e.g., at least about 20%, about 30%, about 40%, about 50%, about 60%, about 70%, about 80%, about 90%, about 95%, about 96%, about 97%, about 98%, about 99%, or more. In certain embodiments, the term refers to a decrease in the number of T cells and/or NK cells and/or KLRG1-expressing cancer cells in a subject or in a sample to an amount below detectable limits.

The term “autoimmune disorder” as used herein refers to any disorder associated with an autoimmune reaction. Examples include, without limitation, multiple sclerosis, Crohn's disease, ulcerative colitis, lupus, and inflammatory bowel disease.

The term “cancer,” refers to any malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from the group of tumor-forming cancers. A person skilled in the art will recognize which cancers fall within the purview of that group.

Another example of a cancer is myelodysplastic syndrome (MDS).

The term “transplant” as used herein refers to a section of tissue, or a complete organ, that is removed from its original natural site or host and transferred to a new position in the same person or in a separate individual. Methods for treating recipients of transplants relates to methods of inhibiting organ or tissue transplant rejection, particularly in mammals. More particularly, the present disclosure relates to methods of inhibiting transplant rejection in mammals in need thereof, which can include administering to such mammals a transplant rejection inhibiting amount of anti-KLRG1 binding agents, including antibodies which specifically bind KLRG1 and fragments thereof.

The term “isolated” as used herein refers to a polypeptide that is substantially free of cellular material, viral material, and/or culture medium (when produced by recombinant DNA techniques), or chemical precursors or other chemicals (when chemically synthesized). Moreover, an “isolated fragment” is a fragment of a polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose. Thus, the term “isolated” refers to a molecule that is substantially free of its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or approximately at least 70-80% (w/w) pure, more preferably, approximately at least 80-90% (w/w) pure, even more preferably, approximately 90-95% pure; and, most preferably, approximately at least 95%, approximately at least 96%, approximately at least 97%, approximately at least 98%, approximately at least 99%, or approximately at least 100% (w/w) pure.

The term “fragment,” as applied to a polypeptide, as used herein refers to an amino acid sequence of reduced length relative to a reference polypeptide or amino acid sequence and comprising, consisting essentially of, and/or consisting of an amino acid sequence of contiguous amino acids identical or almost identical (e.g., approximately 90%, approximately 92%, approximately 95%, approximately 98%, approximately 99% identical) to the reference polypeptide or amino acid sequence. Such a polypeptide fragment according to the present disclosure may be, where appropriate, included in a larger polypeptide of which it is a constituent. In some embodiments, such fragments can comprise, consist essentially of, and/or consist of peptides having a length of at least about 4, about 5, about 6, about 8, about 10, about 12, about 15, about 20, about 25, about 30, about 35, about 40, about 45, about 50, about 100, about 150, about 200, or more consecutive amino acids of a polypeptide or amino acid sequence according to the present disclosure.

The terms “protein” and “polypeptide” as used herein are used interchangeably and refer to and encompass both peptides and proteins, unless indicated otherwise.

A “fusion protein” as used herein refers to a polypeptide produced when two heterologous nucleotide sequences or fragments thereof coding for two (or more) different polypeptides not found fused together in nature are fused together in the correct translational reading frame. Illustrative fusion polypeptides include fusions of a polypeptide of the present disclosure (or a fragment thereof) to all or a portion of glutathione-transferase, maltose-binding protein, or a reporter protein (e.g., Green Fluorescent Protein, 0-glucuronidase, 0-galactosidase, luciferase, etc.), hemagglutinin, c-Myc, FLAG epitope, and the like.

The terms “antibody” or “antibodies” as used herein refers to all types of immunoglobulins, including IgG, IgM, IgA, IgD, and IgE. The antibody can be monoclonal or polyclonal and can be of any species of origin, including, for example, mouse, rat, rabbit, horse, goat, sheep, camel, human, humanized, or can be a chimeric antibody. The antibodies can be recombinant monoclonal antibodies produced according to the methods disclosed, for example, in U.S. Pat. No. 4,474,893 or U.S. Pat. No. 4,816,567. The antibodies can also be chemically constructed, for example, according to the methods disclosed in U.S. Pat. No. 4,676,980.

The terms “antigen-binding domain,” “antigen-binding fragment,” and “binding fragment” as used herein refer to a part of an antibody molecule that comprises amino acids responsible for the specific binding between the antibody and the antigen. In instances where an antigen is large, the antigen-binding domain may only bind to a part of the antigen. A portion of the antigen molecule that is responsible for specific interactions with the antigen-binding domain is referred to as “epitope” or “antigenic determinant.”

The phrase “disorder associated with KLRG1,” as used herein, refers to any disease, disorder, or condition in which KLRG1 protein and/or expression of KLRG1, plays a role in the cause, pathology, side effect, symptom, or other aspect in the disease, disorder, or condition. Examples of such disorders include, without limitation, autoimmune disorders (e.g., inclusion body myositis (IBM), multiple sclerosis, and rheumatoid arthritis), transplantation disorders, type 1 diabetes, and cancers (e.g., melanomas, prostate cancers, and certain leukemias and lymphomas, such as mature T and NK cell lymphomas and T cell large granular lymphocytic leukemia (T-LGLL))). Other examples of disorders associated with KLRG1 include, but are not limited to, infections with microbes (e.g. bacteria), viruses (e.g., systemic viral infections such as influenza, viral skin diseases such as herpes or shingles), or parasites.

Another example of a disorder associated with KLRG1 is myelodysplastic syndrome (MDS).

The term “KLRG1 activity” as used herein refers to one or more lymphocyte co-inhibitory activities associated with KLRG1. For example, KLRG1 activity may mean modulation of cytotoxic T and NK cell activation.

The term “modulate,” and its cognates, as used herein refer to a reduction or an increase in the activity of KLRG1 associated with activation of T cells and NK cells due to its interaction with an anti-KLRG1 antibody, wherein the reduction or increase is relative to the activity of KLRG1 in the absence of the same antibody. A reduction or an increase in activity is preferably at least about 10%, about 20%, about 30%, about 40%, about 50%, about 60%, about 70%, about 80%, about 90%, or more. When KLRG1 activity is reduced, the terms “modulatory” and “modulate” are interchangeable with the terms “inhibitory” and “inhibit.” When KLRG1 activity is increased, the terms “modulatory” and “modulate” are interchangeable with the terms “activating” and “activate.”

### Antibodies and Compositions

Antibodies, such as those described herein, exhibit at least two functions in the immune system. They bind antigens, e.g., KLRG1, and eliminate these antigens, including cells expressing the antigen, via the immunoglobulin effector functions, including but not limited to activation of the complement system or interaction with cellular receptors (Fc receptors) on phagocytic cells such as macrophages, and/or other immune cells such as NK cells, leukocytes, platelets, and placental trophoblasts. Disclosed herein are antibodies, or fragments thereof, that specifically bind to an extracellular domain of KLRG1 but do not compete with E-cadherin, N-cadherin, or R-cadherin binding to KLRG1, and deplete cells expressing KLRG1 when administered to a subject.

Antibody-dependent cellular phagocytosis (ADCP), antibody-dependent cellular cytotoxicity (ADCC), and complement-dependent cytotoxicity (CDC) are three well known antibody mediated mechanisms for killing, and thus depleting, target cells.

Though not wishing to be bound by mechanism or theory, binding of the antibody to the target cell through the antigen binding region (variable domain) of the antibody can provide a linkage of the target cell to immune effectors through the Fc region(s) of the constant region of the antibody. In ADCC, typically the Fc region of the antibody binds to FcγRIIIa receptor on the immune effector cell, e.g., an NK cell, which can then kill the target cell. In ADCP, typically the Fc region of the antibody binds to FcγRIIa receptor on the immune effector cell, e.g., a macrophage cell, which can then engulf and kill the target cell. CDC is induced when the immune complex C1q binds to the Fc region of the antibody bound to the target cell, triggering the formation of a membrane attack complex that punches holes into the surface of the target cell.

Thus, the constant region of the antibody mediates effector functions, including the activation of complement and interaction with Fc receptors, enabling effects such as ADCC, ADCP, or CDC. Neither CH1 nor Cκ or Cλ domains mediate effector functions, which is the reason why Fabs do not show ADCC, ADCP, or CDC.

There are three classes of Fe gamma receptors, FcγRI (CD64), FcγRII (CD32), and FcγRIII (CD16). Only FcγRI is able to bind IgG in a monomeric form, and the affinity of FcγRI receptors compared to the immunoglobulin receptors FcγRII and FcγRIII is high. The high affinity receptor FcγRI is constitutively expressed on monocytes, macrophages, and dendritic cells, and expression can be induced on neutrophils and eosinophils. Thus, these cells can be recruited to a target cell through antibody or antibody fragment thereof comprising Fc region, bound to the target cell.

The FcγRIIa receptor is found on macrophages, monocytes, and neutrophils, and the FcγRIIb receptor is found on B-cells, macrophages, mast cells, and eosinophils. The FcγRIIIa receptor is found on NK cells, macrophages, eosinophils, monocytes, and T cells, and the FcγRIIIb receptor is highly expressed on neutrophils. Again, these various cell types can be recruited to a target cell by an antibody bound to the target cell through an antibody or antibody fragment thereof comprising the fc region, bound to the target cell.

Thus, in some aspects the KLRG1 binding molecules, including antibodies and antigen binding fragments thereof disclosed herein, comprise a KLRG1 antigen binding site together with an antibody constant domain or fragment thereof. This can function to mediate an effector function, including but not limited to ADCC, ADCP, or CDC. In some aspects the KLRG1 binding molecule consists of or comprises the antigen binding site of an antibody and a peptide binding Fc-effector molecules, as described in International Patent Application Publication No. WO 02/44215.

Intact antibodies, also known as immunoglobulins, are typically tetrameric glycosylated proteins composed of two light (L) chains of approximately 25 kilodaltons (kDa) each and two heavy (H) chains of approximately 50 kDa each. An exemplary carbohydrate moiety with which antibodies may be glycosylated is a fucose moiety. Two types of light chain, designated as the λ chain and the κ chain, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2.

The subunit structures and three-dimensional configurations of different classes of immunoglobulins are well known in the art. Briefly, each light chain can be composed of an N-terminal variable domain (VL) and a constant domain (CL). Each heavy chain can be composed of an N-terminal variable domain (VH), three or four constant domains (CH), and a hinge region. The CH domain most proximal to VH is designated as CH1. The VH and VL domains consist or comprise of four regions of relatively conserved sequence called framework regions (FR1, FR2, FR3, and FR4), which form a scaffold for three regions of hypervariable sequence called CDRs. The CDRs can contain most of the residues responsible for specific interactions with the antigen. The three CDRs are referred to as CDR1, CDR2, and CDR3. CDR constituents on the heavy chain are referred to as H1, H2, and H3, while CDR constituents on the light chain are referred to as L1, L2, and L3, accordingly. CDR3 and particularly H3, are the greatest source of molecular diversity within the antigen-binding domain. H3, for example, can be as short as two amino acid residues of greater than 26.

Antibody fragments included within the scope of the present disclosure include, for example: Fab, Fab′, F(ab′)2, and Fv fragments; domain antibodies, diabodies; vaccibodies, linear antibodies; single-chain antibody molecules; and multi-specific antibodies formed from antibody fragments. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (see Huse et al, Science 1989 Dec. 8; 246(4935):1275-1281).

The Fab fragment (Fragment antigen-binding), or Fab, consists or comprises of the VH-CH1 and VL-CL domains covalently linked by a disulfide bond between the constant regions. Known to those skilled in the art, a Fab (˜50 kDa in size) is a monovalent fragment that is produced from IgG and IgM, consisting or comprising of the VH, CH1 and VL, CL regions, linked by an intramolecular disulfide bond. To overcome the tendency of non-covalently linked VH and VL domains in the Fv to dissociate when co-expressed in a host cell, a single chain (sc) Fv fragment (scFv) can be constructed. In a scFv, a flexible and adequately long polypeptide links either the C-terminus of the VH to the N-terminus of the VL, or the C-terminus of the VL to the N-terminus of the VH. Most commonly, a 15-residue (Gly4Ser)₃ peptide (SEQ ID NO: 15) can be used as a linker, but other linkers are also known in the art.

Antibody diversity is a result of combinatorial assembly of multiple germline genes encoding variable regions and a variety of somatic events. The somatic events can include recombination of variable gene segments with diversity (D) and joining (J) gene segments to make a complete VH region and the recombination of variable and joining gene segments to make a complete VL region. The recombination process itself is imprecise, resulting in the loss or addition of amino acids at the V(D)J junctions. These mechanisms of diversity occur in the developing B cell prior to antigen exposure. After antigenic stimulation, the expressed antibody genes in B cells can undergo somatic mutation.

Based on the estimated number of germline gene segments, the random recombination of these segments, and random VH-VL pairing, up to approximately 1.6×10⁷ different antibodies can be produced according to Fundamental Immunology, 3rd ed., ed. Paul, Raven Press, New York, N.Y., 1993. When other processes which contribute to antibody diversity (such as somatic mutation) are considered, it is thought that upwards of approximately 1×10¹⁰ different antibodies could be potentially generated, as supported by Immunoglobulin Genes, 2nd ed., eds. Jonio et al., Academic Press, San Diego, Calif, 1995. Because of the many processes involved in antibody diversity, it is highly unlikely that independently generated antibodies will have identical amino acid sequences in the CDRs and heavy and light chain variable regions.

The present disclosure provides a novel variable heavy chain and full heavy chain sequences, which are effective in depleting cells expressing cell surface KLRG1. The scaffold structure for carrying a CDR can generally be, though is not limited to, an antibody heavy or light chain, or a portion thereof, in which the CDR is located at a location corresponding to the CDR of naturally occurring VH and VL. The structures and locations of immunoglobulin variable domains may be determined, for example, as described in Kabat et al., Sequences of Proteins of Immunological Interest, No. 91-3242, National Institutes of Health Publications, Bethesda, Md., 1991.

In certain embodiments, the antibody, or fragment thereof, comprises a heavy chain variable region that includes SEQ ID NO:4, and a light chain variable region that comprises three light chain CDRs SEQ ID NO:11 (CDR-L1), SEQ ID NO:12 (CDR-L2), and SEQ ID NO:13 (CDR-L3). In some aspects of these embodiments, the antibody, or fragment thereof, comprises a heavy chain variable region comprising SEQ ID NO:4, and a light chain variable region comprising SEQ ID NO:5. In some aspects of these embodiments, the antibody or fragment thereof, comprises a heavy chain comprising SEQ ID NO:6. Alternatively, or additionally, the antibody, or fragment thereof, comprises a light chain comprising SEQ ID NO:7. In more specific aspects of these embodiments, the antibody, or fragment thereof, comprises a heavy chain comprising SEQ ID NO:6, and a light chain comprising SEQ ID NO:7.

TABLE 1
The amino acid sequence of human KLRG1 ECD (SEQ ID NO: 1), cyno KLRG1
ECD (SEQ ID NO: 2) and human E-cadherin ECD (SEQ ID NO: 3), with epitope amino acid
sequence PLNFSRI (SEQ ID NO: 14) underlined in human and cyno KLRG1.

| | |
|---|---|
| human KLRG1 | LCQGSNYSTCASCPSCPDRWMKYGNHCYYFSVEEKDWNSSLEFCLARDSHLLV |
| extracellular domain | ITDNQEMSLLQVFLSEAFCWIGLRNNSGWRWEDGSPLNFSRISSNSFVQTCGA |
| (ECD) (SEQ ID NO: 1), | INKNGLQASSCEVPLHWVCKKVRL |
| cyno KLRG1 extracellular | LCQGSKYSTCASCPSCPDHWMKYGNHCYYFSVEKKDWISSLEFCLARDSHLLM |
| domain (ECD) (SEQ ID | ITDKQEMSLLQDFLSEAFHWVGLRNNSGWRWEDGSPLNFSRIYSNSLVQTCG |
| NO: 2), | AINKNSLQASSCEVSLQWVCKKVSP |
| human E-cadherin full | DWVIPPISCPENEKGPFPKNLVQIKSNKDKEGKVFYSITGQGADTPPVGVFIIER |
| length (SEQ ID NO: 3) | ETGWLKVTEPLDRERIATYTLFSHAVSSNGNAVEDPMEILITVTDQNDNKPEFT |
| | QEVFKGSVMEGALPGTSVMEVTATDADDDVNTYNAAIAYTILSQDPELPDKN |
| | MFTINRNTGVISVVTTGLDRESFPTYTLVVQAADLQGEGLSTTATAVITVTDTN |
| | DNPPIFNPTTYKGQVPENEANVVITTLKVTDADAPNTPAWEAVYTILNDDGGQ |
| | FVVTTNPVNNDGILKTAKGLDFEAKQQYILHVAVTNVVPFEVSLTTSTATVTVD |
| | VLDVNEAPIFVPPEKRVEVSEDFGVGQEITSYTAQEPDTFMEQKITYRIWRDTA |
| | NWLEINPDTGAISTRAELDREDFEHVKNSTYTALIIATDNGSPVATGTGTLLLILS |
| | DVNDNAPIPEPRTIFFCERNPKPQVINIIDADLPPNTSPFTAELTHGASANWTIQ |
| | YNDPTQESIILKPKMALEVGDYKINLKLMDNQNKDQVTTLEVSVCDCEGAAGV |
| | CRKAQPVEAGLQIPAILGILGGILALLILILLLLLFLRRRAVVKEPLLPPEDDTRDNV |
| | YYYDEEGGGEEDQDFDLSQLHRGLDARPEVTRNDVAPTLMSVPRYLPRPANP |
| | DEIGNFIDENLKAADTDPTAPPYDSLLVFDYEGSGSEAASLSSLNSSESDKDQDY |
| | DYLNEWGNRFKKLADMYGGGEDD |

TABLE 2
Amino acid sequences of CDRs and heavy and light chain variable regions for anti-
KLRG1 antibodies.

| ABC008 VH | QVQLVQSGAEVKKPGASVKVSCKASGYTFTDHNMHWVKQATGQGLEWFGFINPNTGVT | |
|---|---|---|
| | RYNQKFQGRVTLTINKAISTAYLELSSLRSEDTAVYYCARDYYGSAWFAYWGQGTLVTVSS | |
| | (SEQ ID NO: 4) | |
| ABC008 VL | DIVMTQSPDSLAVSLGERATINCKSSQTLLYSSDQKNYLAWYQQKPGQPPKLLIYWASTRE | |
| | SGVPDRFSGSGSGTDFTLTISSLQAEDVAVYYCQQYYNYPTFGGGTKVEIK (SEQ ID NO: 5) | |
| ABC008 HC | QVQLVQSGAEVKKPGASVKVSCKASGYTFTDHNMHWVKQATGQGLEWFGFINPNTGVT | |
| | RYNQKFQGRVTLTINKAISTAYLELSSLRSEDTAVYYCARDYYGSAWFAYWGQGTLVTVSS | |
| | ASTKGPSVFPLAPSSKSTSGGTAALGCLVKDYFPEPVTVSWNSGALTSGVHTFPAVLQSSGL | |
| | YSLSSVVTVPSSSLGTQTYICNVNHKPSNTKVDKKVEPKSCDKTHTCPPCPAPELLGGPSVFL | |
| | FPPKPKDTLMISRTPEVTCVVVDVSHEDPEVKFNWYVDGVEVHNAKTKPREEQYNSTYRV | |
| | VSVLTVLHQDWLNGKEYKCKVSNKALPAPIEKTISKAKGQPREPQVYTLPPSREEMTKNQV | |
| | SLTCLVKGFYPSDIAVEWESNGQPENNYKTTPPVLDSDGSFFLYSKLTVDKSRWQQGNVFS | |
| | CSVMHEALHNHYTQKSLSLSPGK (SEQ ID NO: 6) | |
| ABC008 LC | DIVMTQSPDSLAVSLGERATINCKSSQTLLYSSDQKNYLAWYQQKPGQPPKLLIYWASTRE | |
| | SGVPDRFSGSGSGTDFTLTISSLQAEDVAVYYCQQYYNYPTFGGGTKVEIKRTVAAPSVFIFP | |
| | PSDEQLKSGTASVVCLLNNFYPREAKVQWKVDNALQSGNSQESVTEQDSKDSTYSLSSTLT | |
| | LSKADYEKHKVYACEVTHQGLSSPVTKSFNRGEC (SEQ ID NO: 7) | |
| ABC008 HC | CDR-H1 | GYTFTDH (SEQ ID NO: 8) |
| | CDR-H2 | NPNTGV (SEQ ID NO: 9) |
| | CDR-H3 | DYYGSAWFAY (SEQ ID NO: 10) |
| ABC008 LC | CDR-L1 | KSSQTLLYSSDQKNYLA (SEQ ID NO: 11) |
| | CDR-L2 | WASTRES (SEQ ID NO: 12) |
| | CDR-L3 | QQYYNYPT (SEQ ID NO: 13) |

In some embodiments, the KLRG1 bound by the disclosed antibodies or fragments thereof can be human KLRG1 or cyno KLRG1. In some embodiments, the antibody, or fragment thereof, can specifically bind the epitope PLNFSRI (SEQ ID NO: 14), or a fragment thereof, comprising at least five (5) contiguous amino acids.

In some embodiments, the antibodies can be expected to retain the specificity of binding and/or the ability to be KLRG11-expressing cell-depleting agents so long as the antibody amino acid sequences comprise a sequence which is at least about 70%, about 75%, about 80%, about 85%, about 90%, about 95%, about 96%, about 97%, about 98%, about 99%, or more identical to SEQ ID NO:4, 5, 6, or 7. In some aspects of these embodiments, the antibody or fragment thereof retains the heavy and light chain CDRs of SEQ ID NOs. 8-13. In each of these aspects, the heavy chain variable region comprises SEQ ID NO:4 and the light chain CDRs of SEQ ID Nos. 11-13. The percent identity can be determined by standard alignment algorithms such as, for example, Basic Local Alignment Tool (BLAST) described in Altshul et al. (1990) J. Mol. Biol., 215: 403-410, the algorithm of Needleman et al. (1970) J. Mol. Biol., 48: 444-453, or the algorithm of Meyers et al. (1988) Comput. Appl. Biosci., 4: 11-17.

In some embodiments, the antibody, or fragment thereof, includes a monoclonal antibody. In some embodiments, the monoclonal antibody, or fragment thereof, includes a chimeric antibody, or a fragment thereof. In some embodiments, the monoclonal antibody, or a fragment thereof, includes a humanized antibody, or a fragment thereof.

In some embodiments, the monoclonal antibody, or fragment thereof, comprises a heavy chain variable region that includes SEQ ID NO:4, and a light chain variable region that comprises three light chain CDRs SEQ ID NO:11 (CDR-L1), SEQ ID NO:12 (CDR-L2), and SEQ ID NO:13 (CDR-L3). In some aspects of these embodiments, the monoclonal antibody, or fragment thereof, comprises a heavy chain variable region that includes SEQ ID NO:4 and a light chain variable region that includes SEQ ID NO:5. In some aspects of these embodiments, the monoclonal antibody, or fragment thereof, comprises a heavy chain that includes SEQ ID NO:6. In some aspects of these embodiments, the monoclonal antibody, or fragment thereof, comprises a light chain that includes SEQ ID NO:7. In some more specific aspects of these embodiments, the monoclonal antibody, or fragment thereof, comprises a heavy chain that includes SEQ ID NO:6, and a light chain that includes SEQ ID NO:7.

In some embodiments, the antibody, or a fragment thereof, comprises a heavy chain variable region that includes SEQ ID NO:4, a heavy chain that includes the amino acid sequence of SEQ ID NO:6, or a sequence having approximately at least 90% sequence identity thereto, e.g., at least about 95%, about 96%, about 97%, about 98%, or about 99% identical thereto, and a light chain comprising light chain CDRs SEQ ID NO: 11 (CDR-L1), SEQ ID NO:12 (CDR-L2), and SEQ ID NO:13 (CDR-L3). In some embodiments, the antibody, or fragment thereof, comprises a heavy chain that includes at least 50 contiguous amino acids of the amino acid sequence of SEQ ID NO:6, or a sequence approximately at least 90% identical thereto, e.g., to at least about 100 or about 150 or about 200 or more contiguous amino acids.

In some embodiments, the antibody, or a fragment thereof, comprises a heavy chain variable region that includes SEQ ID NO:4, a light chain that includes the amino acid sequence of SEQ ID NO:7, or a sequence having approximately at least 90% sequence identity thereto, e.g., at least about 95%, about 96%, about 97%, about 98%, or about 99% identical thereto. In some embodiments, the antibody, or fragment thereof, comprises a light chain that includes at least 50 contiguous amino acids of the amino acid sequence of SEQ ID NO:7, or a sequence approximately at least 90% identical thereto, e.g., to at least about 100 or about 150 or about 200 or more contiguous amino acids.

In some embodiments, the antibody, or a fragment thereof, comprises a heavy chain variable region that includes SEQ ID NO:4, a heavy chain that includes the amino acid sequence of SEQ ID NO:6, or a sequence having approximately at least 90% sequence identity thereto, e.g., at least about 95%, about 96%, about 97%, about 98%, or about 99% identical thereto, and comprises a light chain that includes the amino acid sequence of SEQ ID NO:7, or a sequence having approximately at least 90% sequence identity thereto, e.g., at least about 95%, about 96%, about 97%, about 98%, or about 99% identical thereto.

### Depletion of KLRG1 Expressing Cells

Disclosed herein are methods of depleting KLRG1-expressing T cells and/or NK cells in a subject in need of treatment. Methods provided for herein include delivering to a subject in need of treatment an effective amount of a KLRG1-depleting agent, such as an antibody, or a fragment thereof, such as those disclosed herein, that specifically bind to the extracellular domain of KLRG1, thereby depleting KLRG1-expressing T cells in the subject.

Also disclosed herein are methods of treating a disorder associated with excess and/or unwanted KLRG1-expressing T cells in a subject in need of treatment in which the method includes delivering to the subject a therapeutically effective amount of a KLRG1-depleting agent (e.g., the anti-KLRG1 antibodies disclosed above). The KLRG1-depleting agent can deplete KLRG1-expressing T cells, with delivery to the subject depleting the excess or unwanted KLRG1-expressing T cells.

In some embodiments of the method, the disorder can be a transplant-related disorder, with the delivery to the subject depleting KLRG1-expressing pathogenic T cells and/or NK cells attacking transplanted tissues in the subject. In some embodiments of the method, the disorder can be an autoimmune disease (e.g., inclusion-body myositis), with delivery to the subject depleting KLRG1-expressing pathogenic T cells and/or NK cells attacking self-tissues in the subject.

Also disclosed herein are methods of treating cancer in a subject, with the cancer including cancer cells that express KLRG1. The methods can include delivering to the subject a therapeutically effective amount of a KLRG1-depleting agent, such as an antibody, or a fragment thereof disclosed herein, that specifically binds to the extracellular domain of KLRG1.

KLRG1, a cell surface marker known to be present on mature cytotoxic T and NK cells, is also present on certain mature T and NK cell lymphomas and leukemias (see Examples 1-8). Methods of treatment described herein relate to this discovery that KLRG1-depleting agents can be useful in the treatment of aggressive NK cell leukemia (ANKL), NK-T-cell lymphoma (NKTCL), hepatosplenic T cell lymphoma (HSTCL), gamma-delta T cell lymphoma (GDTCL), among other certain mature T and NK cell lymphomas/leukemias. For example, methods of treatment can include administering to a subject in need thereof an effective amount of a KLRG1 depleting agent (e.g., a KLRG1-expressing-cell depleting agent) including the antibodies, or fragments thereof, as described herein. Additionally, an antibody drug conjugate (ADC) of the antibodies or fragments thereof described herein can eliminate or reduce the number of neoplastic T or NK cells. The ADCs disclosed herein may comprise a toxic agent or another therapeutic agent as the conjugated drug, as described below.

In some embodiments, certain leukemias and lymphomas that involve the proliferation of cells that express KLRG1 are treated. KLRG1 is normally expressed on subpopulations of T and NK cells, particularly mature T and NK cells. Some hematological neoplasms involve the proliferation of mature T and NK cells, so that these tumor cells often express KLRG1. Exemplary leukemias and lymphomas that may be treated include T-cell prolymphocytic leukemia, T-cell large granular lymphocytic leukemia, chronic lymphoproliferative disorder of NK cells, aggressive NK-cell leukemia, systemic EBV+ T-cell lymphoma of childhood, hydroa vacciniforme-like lymphoproliferative disorder, adult T-cell leukemia/lymphoma, NK/T-cell lymphoma, enteropathy-associated T-cell lymphoma, monomorphic epitheliotropic intestinal T-cell lymphoma, indolent T-cell lymphoproliferative disorder of the GI tract, hepatosplenic T-cell lymphoma, subcutaneous panniculitis-like T-cell lymphoma, mycosis fungoides, Sezary syndrome, primary cutaneous CD30+ T-cell lymphoproliferative disorders, lymphomatoid papulosis, primary cutaneous anaplastic large cell lymphoma, primary cutaneous gamma-delta T-cell lymphoma, primary cutaneous CD8+ aggressive epidermotropic cytotoxic T-cell lymphoma, primary cutaneous acral CD8+ T-cell lymphoma, primary cutaneous CD4+ small/medium T-cell lymphoproliferative disorder, peripheral T-cell lymphoma, NOS, angioimmunoblastic T-cell lymphoma, follicular T-cell lymphoma, nodal peripheral T-cell lymphoma with TFH phenotype, anaplastic large-cell lymphoma ALKT+, anaplastic large-cell lymphoma ALK−, and breast implant-associated anaplastic large-cell lymphoma.

In particular, aggressive NK cell leukemia (ANKL) and NK/T-cell lymphoma (NKTCL) are related disorders that may constitute a spectrum of disease and may be treated by administration of the antibodies disclosed herein. Hepatosplenic T cell lymphoma (HSTCL) and gamma-delta T cell lymphoma (GDTCL), which overlaps with HSTCL, as some HSTCL express gamma/delta T cell receptors, may also be treated using the antibodies and methods disclosed herein.

Disclosed herein are adjunct therapies for treatment of cancer in a subject, such as a subject that is undergoing checkpoint therapy. The adjunct therapies can be performed regardless of whether the cancer expresses KLRG1. The adjunct therapies can include delivering to the subject a therapeutically effective amount of a KLRG1-depleting agent, such an antibody, or a fragment thereof, that specifically binds to the extracellular domain of KLRG1. Delivery to the subject can modulate KLRG1 activity by depleting KLRG1-expressing pathogenic T cells and/or NK cells attacking self-tissues in the subject.

Disclosed herein are methods of depleting KLRG1-expressing cells in a mixed population of cells. The KLRG1-expressing cells can include one or more cells selected from a group consisting of T cells and/or NK cells and/or cancer cells. The method can include delivering to the mixed population of cells an effective amount of a KLRG1-depleting agent, such as an antibody, or a fragment thereof, that specifically binds to KLRG1 and depletes KLRG1-expressing T cells and/or NK cells and/or cancer cells, thereby depleting KLRG1-expressing T cells and/or NK cells and/or cancer cells in the mixed population of cells.

Disclosed herein are methods of selectively depleting KLRG1-expressing CD8 effector T cells, with relative sparing of naïve T cells and/or regulatory T cells. The methods can include delivering to a subject a therapeutically effective amount of an antibody, or fragment thereof, that specifically binds to an extracellular domain of KLRG1, thereby selectively depleting KLRG1-expressing CD8 effector T cells.

Disclosed herein are methods of selectively depleting KLRG1-expressing T cells and/or NK cells in a subject in need thereof. The method can include delivering to the subject a therapeutically effective amount of an antibody, or fragment thereof, that specifically binds to an extracellular domain of KLRG1, thereby depleting KLRG1-expressing T cells and/or NK cells in the subject.

### Immunotoxins as Means for Depletion of KLRG1 Expressing Cells

In some aspects, the antibodies and/or antigen binding fragments thereof provided for by the instant disclosure are conjugated to a toxic agent, and thus do not necessarily rely on endogenous effector cells in ADCC, ADCP, or CDC to deplete the target cells, e.g., pathogenic cells and/or cancer cells expressing cell surface KLRG1.

Immunoconjugates which include one or more cytotoxins are referred to as “immunotoxins.” Antibodies conjugated to a cytotoxic agent, drug, or the like are also known as antibody-drug conjugates (ADC). An immunoconjugate may have a half-life of sufficient periods of time for the antibody-drug conjugate to be internalized, degraded, and induce cell killing by the released toxin. A cytotoxin or cytotoxic agent can include any agent that is detrimental to (e.g., kills) cells. Suitable cytotoxic agents for forming immunoconjugates of the present disclosure include taxol, tubulysins, duostatins, cytochalasin B, gramicidin D, ethidium bromide, emetine, mitomycin, etoposide, tenoposide, vincristine, vinblastine, colchicin, doxorubicin, daunorubicin, dihydroxy anthracin dione, maytansine or an analog or derivative thereof, mitoxantrone, mithramycin, actinomycin D, 1-dehydrotestosterone, glucocorticoids, procaine, tetracaine, lidocaine, propranolol, and puromycin; calicheamicin or analogs or derivatives thereof, antimetabolites (such as methotrexate, 6-mercaptopurine, 6-thioguanine, cytarabine, fludarabin, 5-fluorouracil, decarbazine, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin; as well as duocarmycin A, duocarmycin SA, CC-1065 (a.k.a. rachelmycin), or analogs or derivatives of CC-1065), dolastatin, auristatin, pyrrolo[2,1-c][1,4]benzodiazepins (PDBs), indolinobenzodiazepine (IGNs) or analogues thereof, antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), anti-mitotic agents (e.g., tubulin-targeting agents), such as diphtheria toxin and related molecules (such as diphtheria A chain and active fragments thereof and hybrid molecules); ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, gelonin, mitogellin, restrictocin, phenomycin, and enomycin toxins. Other suitable conjugated molecules include antimicrobial/lytic peptides such as CLIP, Magainin 2, mellitin, Cecropin, and P18; ribonuclease (RNase), DNase I, Staphylococcal enterotoxin-A, pokeweed antiviral protein, diphtheria toxin, and Pseudomonas endotoxin.

### Supplemental Therapeutic Agents

The antibodies of the present disclosure, including fragments thereof and conjugates thereof, can optionally be delivered to a patient in conjunction with other therapeutic agents. The additional therapeutic agents can be delivered concurrently with the antibodies of the present disclosure. As used herein, the word “concurrently” means sufficiently close in time to produce a combined effect (that is, concurrently can be simultaneously, or it can be two or more events occurring within a short time period before or after each other). In certain embodiments, the other therapeutic agent may be conjugated to the antibody or fragment disclosed herein to form an ADC.

In some embodiments the antibodies of the present disclosure can be administered in conjunction with anti-cancer agents, such as: vinca alkaloids (e.g., vinblastine, vincristine); epipodophyllotoxins (e.g., etoposide and teniposide); antibiotics (e.g., dactinomycin (actinomycin D), daunorubicin (daunomycin; rubidomycin), doxorubicin, bleomycin, plicamycin (mithramycin), and mitomycin (mitomycin C)); enzymes (e.g., L-asparaginase); biological response modifiers (e.g., interferon-alfa); platinum coordinating complexes (e.g., cisplatin and carboplatin); anthracenediones (e.g., mitoxantrone); substituted ureas (e.g., hydroxyurea); methylhydrazine derivatives (e.g., procarbazine (N-methylhydrazine; MIH)); adrenocortical suppressants (e.g., mitotane (o,p′-DDD) and aminoglutethimide); adrenocorticosteroids (e.g., prednisone); progestins (e.g., hydroxyprogesterone caproate, medroxyprogesterone acetate, and megestrol acetate); estrogens (e.g., diethylstilbestrol and ethinyl estradiol); antiestrogens (e.g., tamoxifen); androgens (e.g., testosterone propionate and fluoxymesterone); antiandrogens (e.g., flutamide); and gonadotropin-releasing hormone analogs (e.g., leuprolide). In some embodiments the antibodies of the present disclosure can be administered in conjunction with anti-angiogenesis agents, such as antibodies to VEGF (e.g., bevacizumab (AVASTIN), ranibizumab (LUCENTIS)) and other promoters of angiogenesis (e.g., bFGF, angiopoietin-1), antibodies to alpha-v/beta-3 vascular integrin (e.g., VITAXIN), angiostatin, endostatin, dalteparin, ABT-510, CNGRC peptide TNF alpha conjugate (“CNGRC” disclosed as SEQ ID NO: 16), cyclophosphamide, combretastatin A4 phosphate, dimethylxanthenone acetic acid, docetaxel, lenalidomide, enzastaurin, paclitaxel, paclitaxel albumin-stabilized nanoparticle formulation (Abraxane), soy isoflavone (Genistein), tamoxifen citrate, thalidomide, ADH-1 (EXHERIN), AG-013736, AMG-706, AZD2171, sorafenib tosylate, BMS-582664, CHIR-265, pazopanib, PI-88, vatalanib, everolimus, suramin, sunitinib malate, XL184, ZD6474, ATN-161, cilengitide, and celecoxib.

In some embodiments, including but not limited to the treatment of an autoimmune disease or in transplantation treatment, the antibodies of the present disclosure can be administered in conjunction with immunosuppressive agents including, for example, cyclosporine A, rapamycin, glucocorticoids, azathioprine, mizoribine, aspirin derivatives, hydroxychloroquine, methotrexate, cyclophosphamide and FK506 (tacrolimus). In certain embodiments, the immunosuppressive agent may be conjugated to the antibody or fragment disclosed herein to form an ADC.

Anti-KLRG1 antibodies may optionally comprise antibody constant regions or parts thereof. For example, a VL domain may have attached, at its C terminus, antibody light chain constant domains including human Cκ or Cλ chains. Similarly, a specific antigen-binding domain based on a VH domain may have attached all or part of an immunoglobulin heavy chain derived from any antibody isotope, e.g., IgG, IgA, IgE, and IgM and any of the isotope sub-classes, which include but are not limited to, IgG1 and IgG4. The DNA and amino acid sequences for the C-terminal fragment of are well known in the art.

The term “repertoire” refers to a genetically diverse collection of nucleotides derived wholly or partially from sequences that encode expressed immunoglobulins. The sequences can be generated by in vivo rearrangement of, e.g., V, D, and J segments for H chains and, e.g., V and J segment for L chains. Alternatively, the sequences may be generated from a cell line by in vitro stimulation, in response to which the rearrangement occurs. Alternatively, part or all of the sequences may be obtained by combining, e.g., unrearranged V segments with D and J segments, by nucleotide synthesis, randomized mutagenesis, and other methods, for example as disclosed in U.S. Pat. No. 5,565,332.

The terms “specific interaction” and “specific binding” refer to two molecules forming a complex that is relatively stable under physiologic conditions. Specific binding can be characterized by a high affinity and a low to moderate capacity, as distinguished from non-specific binding, which usually has a low affinity with a moderate to high capacity.

Typically, binding is considered specific when the affinity constant KA is higher than approximately 10⁶ M⁻¹, or more preferably higher than approximately 10⁸ M⁻¹. If necessary, non-specific binding can be reduced without substantially affecting specific binding, for example, by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

In certain embodiments, the antibodies can specifically bind an epitope within the extracellular domain (ECD) of human or mouse or monkey KLRG1, with an affinity, as expressed in KD, of at least about 2 nM, about 1 nm, about 100 pM, about 10 pM, or about 5 pM. The amino acid sequences of ECDs of human and cynomolgus KLRG1 are set out in SEQ ID NO:1 and SEQ ID NO:2, as listed in Table 1.

### Methods for Generating Antibodies and Fragments Thereof

This disclosure also provides methods for obtaining an antibody specific for KLRG1. CDRs in such antibodies are not limited to the specific sequences of VH and VL disclosed herein and may include variants of these sequences. Such variants may be derived from the sequences provided herein by a skilled artisan using techniques well known in the art. For example, amino acid substitutions, deletions, or additions, can be made in the framework regions (FRs) and/or in CDRs. While changes in the FRs can usually be designed to improve stability and immunogenicity of the antibody, changes in the CDRs can typically be designed to increase affinity of the antibody for its target.

Changes to FRs include, but are not limited to, humanizing a non-human derived or engineering certain framework residues that are important for antigen contact or for stabilizing the binding site, e.g., changing the class or subclass of the constant region, changing specific amino acid residues which might alter the effector function such as Fc receptor binding, e.g., as described in U.S. Pat. Nos. 5,624,821 and 5,648,260 and Lund et al. (1991) J. Immun. 147: 2657-2662 and Morgan et al. (1995) Immunology 86: 319-324, or changing the species from which the constant region is derived.

Variants of FRs also include naturally occurring immunoglobulin allotypes. Such affinity-increasing changes may be determined empirically by routine techniques that involve altering the CDR and testing the affinity antibody for its target. For example, conservative amino acid substitutions can be made within any one of the disclosed CDRs. Various alterations can be made according to the methods described, for example, in Antibody Engineering, 2nd ed., Oxford University Press, ed. Borrebaeck, 1995. These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class (i.e., nonpolar, polar neutral, basic, or acidic) to which the amino acid belongs.

In one embodiment, substitutions may be chosen from the exemplary conservative substitutions listed in Table 3.

TABLE 3
Exemplary conservative substitutions:

| | Conservative |
|---|---|
| Residue | substitution |
| Ala (A) | Ser (S) |
| Arg (R) | Lys (K) |
| Asn (N) | Gln (Q); His (H) |
| Asp (D) | Glu (E) |
| Cys (C) | Ser (S) |
| Gln (Q) | Asn (N) |
| Glu (E) | Asp (D) |
| Gly (G) | Pro (P) |
| His (H) | Asn (N), Gln (Q) |
| Ile (I) | Leu (L), Val (V) |
| Leu (L) | Ile (I), Val (V) |
| Lys (K) | Arg (R), Gln (Q) |
| Met (M) | Leu (L), Ile (I) |
| Phe (F) | Met (M), Leu (L), Tyr (Y) |
| Ser (S) | Thr (T); Gly (G) |
| Thr (T) | Ser (S), Val (V) |
| Trp (W) | Tyr (Y) |
| Tyr (Y) | Trp (W), Phe (F) |
| Val (V) | Ile (I), Leu (L) |
| Pro (P) | — |

Furthermore, any native residue in the polypeptide may also be substituted with alanine (see, e.g., MacLennan et al. (1998) Acta Physiol. Scand. Suppl. 643:55-67; Sasaki et al. (1998) Adv. Biophys. 35:1-24).

Antibodies of the present disclosure may be altered or mutated for compatibility with species other than the species in which the antibody was produced. For example, antibodies may be humanized or camelized. Humanized forms of non-human (e.g., murine) antibodies are chimeric immunoglobulins, immunoglobulin chains or fragments thereof (such as Fv, Fab, Fab1, F(ab′)2 or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin can be replaced by corresponding non-human residues. Humanized antibodies may also comprise residues which are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin and all or substantially all of the framework (FR) regions (i.e., the sequences between the CDR regions) are those of a human immunoglobulin consensus sequence. The humanized antibody optimally also can comprise at least a portion of an immunoglobulin constant region (Fc), typically that of a human immunoglobulin.

In certain embodiments, the VH and/or VL domains may be germlined, i.e., the framework regions (FRs) of these domains are mutated using conventional molecular biology techniques to match those produced by the germline cells. In other embodiments, the framework sequences remain diverged from the consensus germline sequences.

Methods for humanizing non-human antibodies are well known in the art. The present disclosure, and any invention(s) provided for herein, is not limited to any particular source, species of origin, or method of production. Generally, a humanized antibody has one or more amino acid residues introduced into it from a source that is non-human. These non-human amino acid residues are often referred to as “import” residues, which are typically taken from an “import” variable domain. Humanization can essentially be performed following the method of Winter and co-workers (Jones et al, Nature 321:522 (1986); Riechmann et al, Nature 332:323 (1988); Verhoeyen et al, Science 239:1534 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues (e.g., all the CDRs or a portion thereof), and possibly some FR residues, are substituted by residues from analogous sites in rodent antibodies.

Human antibodies can also be produced using various techniques known in the art, including phage display libraries (Hoogenboom and Winter, J. Mol. Biol. 227:381 (1991); Marks et al, J. Mol Biol 222:581 (1991)). The techniques of Cole et al and Boerner et al are also available for the preparation of human monoclonal antibodies (Cole et al, Monoclonal Antibodies and Cancer Therapy, Alan R. Liss, p. 77 (1985) and Boemer et al, J. Immunol. 147:86 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 9,434,782, 9,253,965, 5,545,807, 5,545,806, 5,569,825, 5,625,126, 5,633,425, and 5,661,016, and in the following scientific publications: Lee, E-Chiang et al. “Complete humanization of the mouse immunoglobulin loci enables efficient therapeutic antibody discovery” Nature Biotechnology volume 32, pages 356-363 (2014); Marks et ah, Bio/Technology 10:779 (1992); Lonberg et 1, Nature 368:856 (1994); Morrison, Nature 368:812 (1994); Fishwild et ah, Nature Biotechnol. 14:M5 (1996); Neuberger, Nature Biotech 14:826 (1 96); Lonberg and Huszar, Intern, Rev. Immunol 13:65 (1995).

Monoclonal antibodies used to carry out the present disclosure can be produced in a hybridoma cell line according to the technique of Kohler and Milstein, Nature 265:495 (1975). For example, a solution containing the appropriate antigen can be injected into a mouse and, after a sufficient time, the mouse sacrificed, and spleen cells obtained. The spleen cells can then be immortalized by fusing them, for example with myeloma cells or with lymphoma cells, typically in the presence of polyethylene glycol, to produce hybridoma cells. The hybridoma cells can then be grown in a suitable medium and the supernatant screened for monoclonal antibodies having the desired specificity. Monoclonal Fab fragments can be produced in E. coli by recombinant techniques known to those skilled in the art. Antibodies specific to the target polypeptide can also be obtained by phage display techniques known in the art.

Various immunoassays can be used for screening to identify antibodies having the desired specificity for the extracellular domain of KLRG1. Numerous protocols for competitive binding or immunoradiometric assays using monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non-interfering epitopes on the polypeptides or peptides of this disclosure can be used as well as a competitive binding assay.

The anti-KLRG1 antibodies described herein can be conjugated to a solid support (e.g., beads, plates, slides, or wells formed from materials such as latex or polystyrene) in accordance with known techniques. Anti-KLRG1 antibodies described herein can likewise be conjugated to detectable groups such as radiolabels (e.g., ³⁵S, ¹²⁵1, ¹³¹I or ⁹⁹mTc, which may also be attached to antibodies using conventional chemistry), enzyme labels (e.g., horseradish peroxidase, alkaline phosphatase), and fluorescence labels (e.g., fluorescein) in accordance with known techniques. Detectable labels further include chemical moieties such as biotin, which may be detected via binding to a specific cognate detectable moiety, e.g., labeled avidin. Determination of the formation of an antibody/antigen complex in the methods of this disclosure can be by detection of, for example, precipitation, agglutination, flocculation, radioactivity, color development or change, fluorescence, luminescence, etc., and is well known in the art.

As described above, anti-KLRG1 antibodies described herein can be linked to another functional molecule, e.g., another peptide or protein (albumin, another antibody, etc.), toxin, radioisotope, cytotoxic or cytostatic agents. For example, the antibodies can be linked to such other functional molecule by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. Nos. 4,640,835, 4,496,689, 4,301,144, 4,670,417, 4,791,192, and 4,179,337. The antibodies can be chemically modified by covalent conjugation to a polymer, for example, to increase their circulating half-life. Exemplary polymers and methods to attach them are also shown in U.S. Pat. Nos. 4,766,106, 4,179,337, 4,495,285, and 4,609,546.

Anti-KLRG1 antibodies described herein may also be altered to have a glycosylation pattern that differs from the native pattern. For example, one or more carbohydrate moieties can be deleted and/or one or more glycosylation sites added to the original antibody. Addition of glycosylation sites to the presently disclosed antibodies may be accomplished by altering the amino acid sequence to contain glycosylation site consensus sequences known in the art. Another means of increasing the number of carbohydrate moieties on the antibodies is by chemical or enzymatic coupling of glycosides to the amino acid residues of the antibody. Such methods are described in International Patent Application Publication No. WO 87/05330, and in Aplin et al. (1981) CRC Crit. Rev. Biochem., 22: 259-306. Reduction of glycosylation can be achieved by removing a glycosylation site by changing one or two amino acids that make up such a site. Removal of any carbohydrate moieties from the antibodies may also be accomplished chemically or enzymatically, for example, as described by Hakimuddin et al. (1987) Arch. Biochem. Biophys., 259: 52; and Edge et al. (1981) Anal. Biochem., 118: 131 and by Thotakura et al.

In one embodiment, the antibody or fragment thereof may contain one or more fucosylated amino acid residues. In another embodiment, the antibody or fragment thereof may lack any fucosylated amino acid residues and/or lack any other glycosylated amino acid residues. The lack of fucosylation may arise from the conditions of manufacture of the antibody or fragment thereof, or may be affected by techniques known to a person of ordinary skill in the art.

In certain embodiments, the monoclonal antibody, or a fragment thereof, can be a chimeric antibody or a humanized antibody. In additional embodiments, the chimeric or humanized antibody comprises at least a portion of the CDRs of the monoclonal antibody. As used herein, a “portion” of a CDR is defined as one or more of the three loops from each of the light and heavy chain that make up the CDRs (e.g., from 1-6 of the CDRs) or one or more portions of a loop comprising, consisting essentially of, or consisting of at least three contiguous amino acids. For example, the chimeric or humanized antibody may comprise 1, 2, 3, 4, 5, or 6 CDR loops, portions of 1, 2, 3, 4, 5, or 6 CDR loops, or a mixture thereof, in any combination.

### Nucleic Acids, Cloning, and Expression Systems

The present disclosure further provides isolated nucleic acids encoding the disclosed antibodies. The nucleic acids may comprise DNA or RNA and may be wholly or partially synthetic or recombinant. Reference to a nucleotide sequence as set out herein encompasses a DNA molecule with the specified sequence and encompasses an RNA molecule with the specified sequence in which U is substituted for T, unless context requires otherwise.

The nucleic acids provided herein can comprise a coding sequence for a CDR, a VH domain, and/or a VL domain, a full heavy chain, and/or a full light chain disclosed herein. The present disclosure also provides constructs in the form of plasmids, vectors, phagemids, transcription or expression cassettes which can include at least one nucleic acid encoding a CDR, a VH domain, and/or a VL domain, a full heavy chain, and/or a full light chain disclosed herein. The disclosure further provides a host cell that may include one or more constructs as above.

Also provided are nucleic acids encoding any CDR (CDR-H1, CDR-H2, CDR-H3, CDR-L1, CDR-L2, or CDR-L3), VH and/or VL domain, heavy chain and/or light chain, as well as methods of making the encoded products. The methods may include expressing the encoded product from the encoding nucleic acid. Expression may be achieved, for example, by culturing under appropriate conditions recombinant host cells containing the nucleic acid. Following production by expression of a VH or VL domain, a specific binding member may be isolated and/or purified using any suitable technique, then used as appropriate and as understood by a person skilled in the art.

Systems for cloning and expression of a polypeptide in a variety of different host cells are well known in the art. For cells suitable for producing antibodies, see Gene Expression Systems, Academic Press, eds. Fernandez et al., 1999. Briefly, suitable host cells include bacteria, plant cells, mammalian cells, and yeast and baculovirus systems. Mammalian cell lines available in the art for expression of a heterologous polypeptide include Chinese hamster ovary (CHO) cells, HeLa cells, baby hamster kidney cells, NS0 mouse myeloma cells, and many others. A common bacterial host is E. coli. Any protein expression system compatible with the present disclosure may be used to produce the disclosed antibodies. Suitable expression systems include transgenic animals described in Gene Expression Systems, Academic Press, eds. Fernandez et al., 1999.

Suitable vectors can be chosen or constructed so that they contain appropriate regulatory sequences, including promoter sequences, terminator sequences, polyadenylation sequences, enhancer sequences, marker genes, and other sequences as appropriate. Vectors may be plasmids or viral, e.g., phage, or phagemid, as appropriate. For further details see, for example, Sambrook et al., Molecular Cloning: A Laboratory Manual, 4th ed., Cold Spring Harbor Laboratory Press, 2012. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, 2nd Edition, eds. Ausubel et al., John Wiley & Sons, 1992.

Further aspects of the disclosure provide a host cell comprising a nucleic acid as disclosed herein, or otherwise derivable from the present disclosures. Still further aspects of the disclosure provide methods that include introducing such nucleic acid into a host cell. The introduction may employ any available technique. For eukaryotic cells, suitable techniques may include calcium phosphate transfection, DEAE-Dextran, electroporation, liposome-mediated transfection, and transduction using retrovirus or other virus, e.g., vaccinia or, for insect cells, baculovirus. For bacterial cells, suitable techniques may include calcium chloride transformation, electroporation, and transfection using bacteriophage. The introduction of the nucleic acid into the cells may be followed by causing or allowing expression from the nucleic acid, e.g., by culturing host cells under conditions for expression of the gene.

### Pharmaceutical Compositions and Methods of Administration

The disclosure provides compositions comprising at least one KLRG1 depletion agent, anti-KLRG1 antibody, and/or fragments thereof, and/or conjugates and/or fusion proteins thereof as described herein. The compositions of the present disclosure can optionally comprise medicinal agents, pharmaceutical agents, carriers, pharmaceutically acceptable carriers, adjuvants, dispersing agents, diluents, and the like. Such compositions may be suitable for pharmaceutical use and administration to patients. By “pharmaceutically acceptable” it is meant a material that is not biologically or otherwise undesirable, i.e., the material can be administered to a subject without causing any undesirable biological effects such as toxicity. The compositions typically comprise one or more antibodies of the present disclosure and a pharmaceutically acceptable excipient. The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser, together with instructions for administration.

A person skilled in the art, with the benefit of the present disclosure in its entirety, will understand various pharmaceutically acceptable carriers can be used, including but not limited to sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide, aluminum hydroxide, and amino acids; alginic acid; pyrogen-free water; isotonic saline; Ringer's solution; ethyl alcohol; phosphate buffer solutions; histidine buffered solutions; and other non-toxic compatible substances employed in pharmaceutical formulations. Pharmaceutically acceptable carriers can include substances that are “generally regarded as safe” (GRAS), for example as designated by the FDA.

The compositions of the present disclosure can be formulated for administration in a pharmaceutical carrier in accordance with known techniques. In the manufacture of a pharmaceutical formulation according to the present disclosure, the compound (including the physiologically acceptable salts thereof) can typically be admixed with, inter alia, an acceptable carrier. The carrier can be a solid or a liquid, or both, and can be formulated with the compound as a unit-dose formulation, for example, a tablet, which can contain from approximately 0.01% or approximately 0.5% to approximately 95% or approximately 99% by weight or by volume of the compound. One or more compounds can be incorporated in the formulations of the present disclosure, which can be prepared by any of the techniques of pharmacy known to those skilled in the art.

A pharmaceutical composition of the present disclosure can be formulated to be compatible with its intended route of administration. Methods to accomplish the administration are known to those skilled in the art. The administration may, for example, be intravenous, intraperitoneal, intramuscular, intracavity, subcutaneous, and/or transdermal. It may also be possible to obtain compositions that may be administered in other manners, including topically or orally, or which may be capable of transmission across mucous membranes.

Solutions or suspensions used for intradermal or subcutaneous application typically include one or more of the following components: a sterile diluent, such as water for injection, saline solution, fixed oils, polyethylene glycols, glycerin, propylene glycol, or other synthetic solvents; antibacterial agents, such as benzyl alcohol or methyl parabens; antioxidants, such as ascorbic acid, or sodium bisulfite; chelating agents, such as ethylenediaminetetraacetic acid; buffers, such as acetates, citrates, or phosphates; and agents for the adjustment of tonicity, such as sodium chloride or dextrose. The pH can be adjusted with acids or bases, such as hydrochloric acid or sodium hydroxide. Such preparations may be enclosed in ampoules, disposable syringes, or multiple dose vials, which can be made, for example, of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.), or phosphate buffered saline (PBS). Typically, the composition should be sterile and should be fluid to the extent that easy syringeability exists. It should be stable under the conditions of manufacture and storage and should be preserved against the contaminating action of microorganisms, such as bacteria and fungi. Prevention of the action of microorganisms can be achieved, for example, by various antibacterial and antifungal agents, including parabens, chlorobutanol, phenol, ascorbic acid, thimerosal, and the like. In many cases, it will be preferable to include in the composition isotonic agents, for example, sugars and/or polyalcohols, such as mannitol, sorbitol, and sodium chloride. The carrier can be a solvent or dispersion medium containing, for example, water, ethanol, polyol (e.g., glycerol, propylene glycol, liquid polyetheylene glycol, and the like), and suitable mixtures thereof. The proper fluidity can be maintained, for example, using a coating, such as lecithin, by the maintenance of the required particle size in the case of dispersion, and/or using surfactants. Prolonged absorption of the injectable compositions can be brought about by including in the composition an agent that delays absorption, for example, aluminum monostearate, and gelatin.

Oral compositions generally include an inert diluent or an edible carrier. They can be enclosed in gelatin capsules or compressed into tablets in some instances. For oral administration, the antibodies can be combined with excipients and used in the form of tablets, troches, or capsules. Pharmaceutically compatible binding agents, and/or adjuvant materials, can be included as part of the composition. The tablets, pills, capsules, troches, and the like can contain any of the following ingredients, or compounds of a similar nature: a binder, such as microcrystalline cellulose, gum tragacanth, or gelatin; an excipient, such as starch or lactose; a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring.

Systemic administration can also be by transmucosal or transdermal means. For transmucosal or transdermal administration, penetrants appropriate to the barrier to be permeated can be used in the formulation. Such penetrants are generally known in the art, and include, for example, detergents, bile salts, and fusidic acid derivatives. Transmucosal administration may be accomplished, for example, using lozenges, nasal sprays, inhalers, or suppositories. For example, in case of antibodies that include the Fc portion, compositions may be capable of transmission across mucous membranes in intestine, mouth, or lungs (e.g., via the FcRn receptor-mediated pathway as described in U.S. Pat. No. 6,030,613). For transdermal administration, the active compounds may be formulated, for example, into ointments, salves, gels, or creams as generally known in the art. For administration by inhalation, the antibodies may be delivered, for example, in the form of an aerosol spray from pressured container or dispenser, which can contain a suitable propellant, e.g., a gas such as carbon dioxide, or a nebulizer.

In certain embodiments, the presently disclosed antibodies can be prepared with carriers that are configured to protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and/or polylactic acid. Methods for preparation of such formulations will be apparent to those skilled in the art. Liposomal suspensions containing the presently disclosed antibodies can also be used as pharmaceutically acceptable carriers. These can be prepared according to methods known to those skilled in the art, for example, as described in U.S. Pat. No. 4,522,811.

It may be advantageous to formulate oral or parenteral compositions in a dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of the composition of the present disclosure can be determined by standard pharmaceutical procedures in cell cultures or experimental animals, e.g., for determining the LD50 (the dose lethal to approximately 50% of the population) and the ED50 (the dose therapeutically effective in approximately 50% of the population). The dose ratio between toxic and therapeutic effects is the therapeutic index and it can be expressed as the ratio LD50/ED50. Compositions that exhibit large therapeutic indices are typically preferred.

For any composition used in the present disclosure, or derivable from the present disclosure, the therapeutically effective dose can be estimated initially from cell culture assays. Examples of suitable bioassays include but are not limited to DNA replication assays, cytokine release assays, transcription-based assays, KLRG1/cadherin binding assays, immunological assays, and other assays, such as those described in the Examples below. The data obtained from the cell culture assays and animal studies can be used in formulating a range of dosage for use in humans. A dose may be formulated in animal models to achieve a circulating plasma concentration range that includes the IC50 (i.e., the concentration of the antibody which achieves a half-maximal inhibition of symptoms). Circulating levels in plasma may be measured, for example, by high performance liquid chromatography. The effects of any particular dosage can be monitored by a suitable bioassay. The dosage lies preferably within a range of circulating concentrations with little or no toxicity. The dosage may vary depending, at least in part, upon the dosage form employed and the route of administration utilized. Alternatively, one can administer the presently disclosed antibodies, or those derivable from the present disclosure, in a local rather than systemic manner, for example, in a depot or sustained-release formulation.

The pharmaceutical composition of any antibody described herein can be in any form that is suitable for intravenous administration.

A further aspect of the present disclosure relates to kits for use in the methods provided for herein or otherwise derivable in view of the present disclosures. A kit can comprise at least one or more antibodies, or fragments thereof, of the present disclosure, and/or one or more antibodies derivable from the present disclosure, in a form suitable for administration to a subject, and/or in a form suitable for compounding into a formulation. The kit can further comprise other components, such as therapeutic agents, carriers, buffers, containers, devices for administration, and the like. The kit can be designed for therapeutic use, diagnostic use, and/or research use, and the additional components can be those suitable for the intended use. A person skilled in the art will recognize various such components suitable for inclusion in kits of this nature. The kit can further comprise labels and/or instructions, e.g., for treatment of a disorder. Such labeling and/or instructions can include, for example, information concerning the amount, frequency, and method of administration of the antibody. A person skilled in the art, in view of the present disclosures, will appreciate the types of instructions that may be included in conjunction as part of the kits. The instructions are provided for herein or are otherwise derivable by a person skilled in the art in view of the present disclosures.

### Exemplification

### Example 1: Hepatosplenic T Cell Lymphoma (HSTCL)

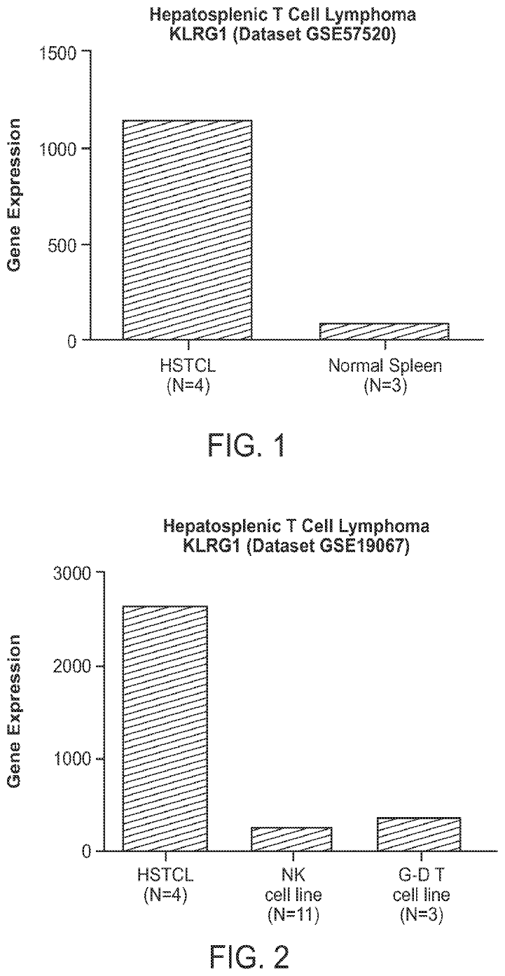

Gene expression analysis of expression data (GSE57520) from tumor biopsies from 4 patients with hepatosplenic T cell lymphoma (HSTCL) was performed and expression compared to normal spleen from 3 patients showing increased expression of KLRG1 (13.0 fold ratio) (FIG. 1). Dataset was obtained from Gene Expression Omnibus database at the National Center for Bioinformatics and analyzed for KLRG1 expression. Accordingly, hepatosplenic T cell lymphoma (HSTCL) is a particularly attractive target for therapies according to the present invention.

### Example 2: Hepatosplenic T Cell Lymphoma (HSTCL)

Gene expression analysis of expression data (GSE19067) from tumor biopsies from 4 patients with hepatosplenic T cell lymphoma (HSTCL) was performed and expression was compared to NK cell lines from 11 and gamma-delta T cell lines from 3 patients showing increased expression of KLRG1 (10.0 fold ratio compared to NK cell lines) (FIG. 2). Dataset was obtained from Gene Expression Omnibus database at the National Center for Bioinformatics and analyzed for KLRG1 expression. Accordingly, hepatosplenic T cell lymphoma (HSTCL) is a particularly attractive target for therapies according to the present invention.

### Example 3: Hepatosplenic T Cell Lymphoma (HSTCL)

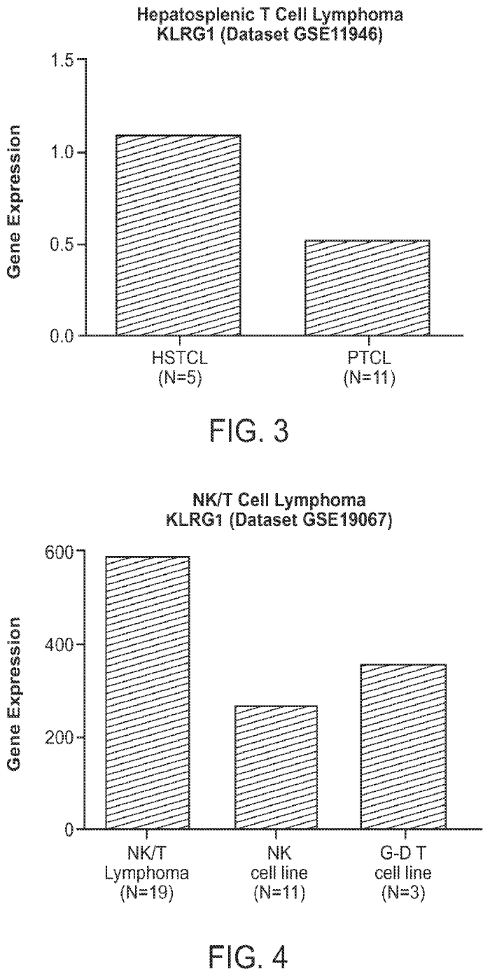

Gene expression analysis of expression data (GSE11946) from tumor biopsies from 5 patients with hepatosplenic T cell lymphoma (HSTCL) was performed and expression was compared to tumor biopsies from 11 patients with other types of peripheral T cell lymphoma (PTCL) showing increased expression of KLRG1 (2.1 fold ratio) (FIG. 3). Dataset was obtained from Gene Expression Omnibus database at the National Center for Bioinformatics and analyzed for KLRG1 expression. Accordingly, hepatosplenic T cell lymphoma (HSTCL) is a particularly attractive target for therapies according to the present invention.

### Example 4: NK/T-cell Lymphoma (NKTCL)

Gene expression analysis of expression data (GSE19067) from tumor biopsies from 19 patients with NK/T-cell lymphoma (NKTCL) was performed and expression was compared to NK cell lines from 11 and gamma-delta T cell lines from 3 patients showing increased expression of KLRG1 (2.2 fold ratio compared to NK cell lines) (FIG. 4). Dataset was obtained from Gene Expression Omnibus database at the National Center for Bioinformatics and analyzed for KLRG1 expression. Accordingly, NK/T-cell lymphoma (NKTCL) is a particularly attractive target for therapies according to the present invention.

### Example 5: Mycosis Fungoides

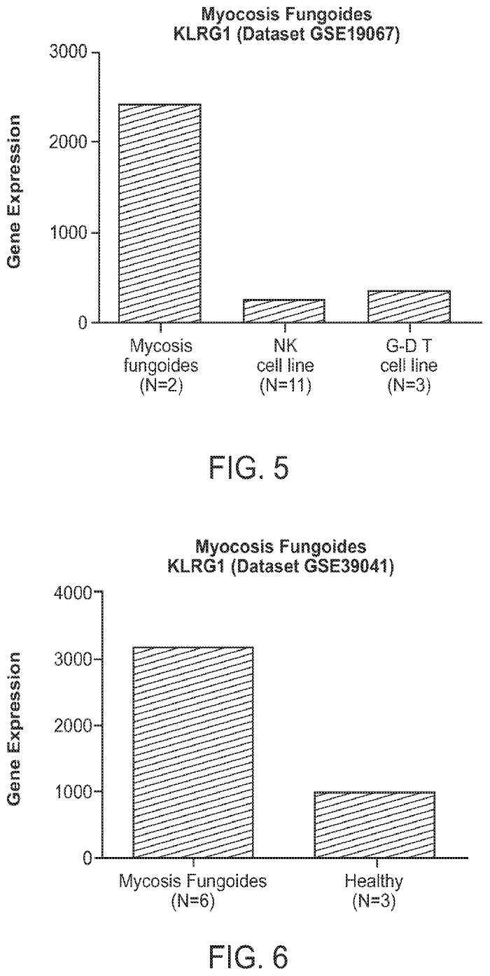

Gene expression analysis of expression data (GSE19067) from tumor biopsies from 2 patients with mycosis fungoides was performed and expression was compared to NK cell lines from 11 and gamma-delta T cell lines from 3 patients showing increased expression of KLRG1 (9.1 fold ratio compared to NK cell lines) (FIG. 5). Dataset was obtained from Gene Expression Omnibus database at the National Center for Bioinformatics and analyzed for KLRG1 expression. Accordingly, mycosis fungoides is a particularly attractive target for therapies according to the present invention.

### Example 6: Mycosis Fungoides

Gene expression analysis of expression data (GSE39041) from tumor biopsies from 6 patients with mycosis fungoides was performed and expression was compared to healthy skin CD4+ T cells from 3 patients showing increased expression of KLRG1 (3.1 fold ratio) (FIG. 6). Dataset was obtained from Gene Expression Omnibus database at the National Center for Bioinformatics and analyzed for KLRG1 expression. Accordingly, mycosis fungoides is a particularly attractive target for therapies according to the present invention.

### Example 7: T and NK Cell Lymphoma Cell Lines

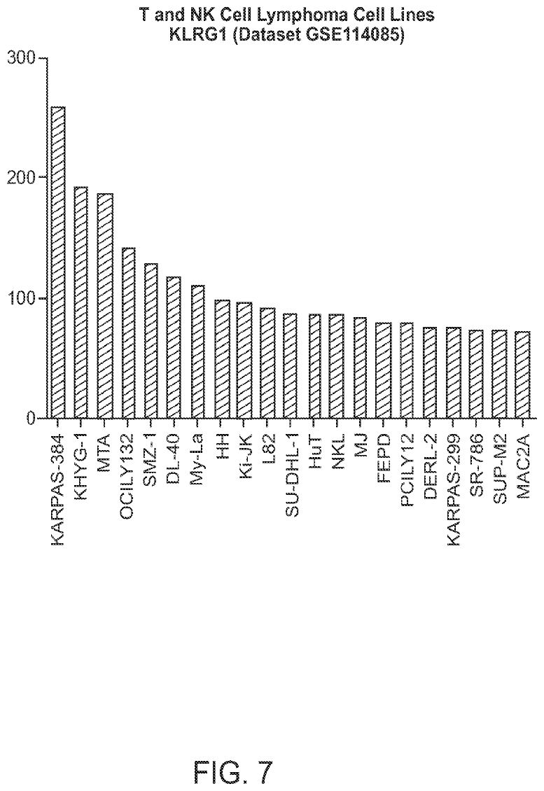

Gene expression analysis of expression data (GSE114085) from T and NK cell lymphoma and leukemia cell lines was performed showing increased expression of KLRG1 in certain cell lines, including KARPAS-384 (gamma-delta T cell line), KHYG-1 (aggressive NK cell leukemia), and MTA (aggressive NK cell leukemia) (FIG. 7). Dataset was obtained from Gene Expression Omnibus database at the National Center for Bioinformatics and analyzed for KLRG1 expression. Accordingly, gamma-delta T cell lymphoma and aggressive NK cell leukemia are particularly attractive targets for therapies according to the present invention.

### Example 8: T Cell Prolymphocytic Leukemia (T-PLL)

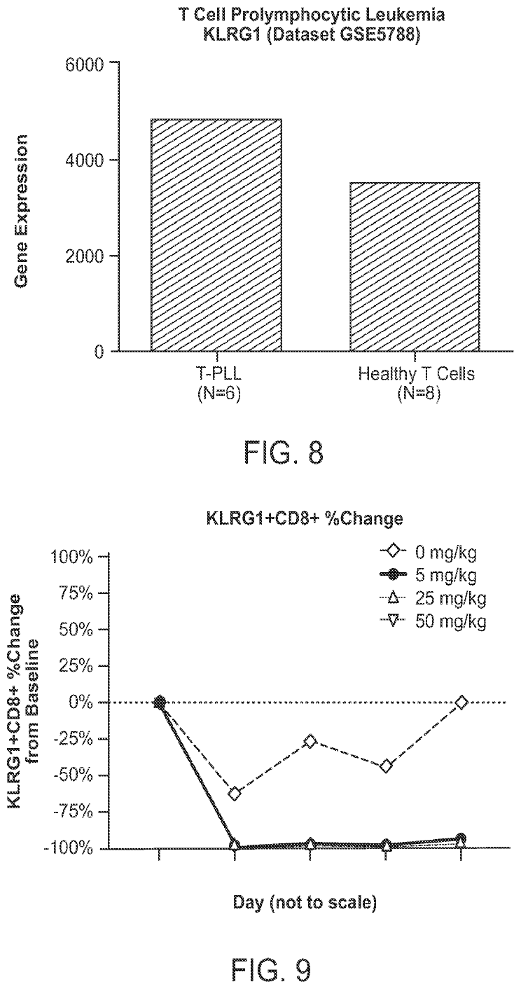

Gene expression analysis of expression data (GSE5788) from tumor biopsies from 6 patients with T cell prolymphocytic leukemia (T-PLL) was performed and expression was compared to healthy donor T cells from 8 patients showing increased expression of KLRG1 (1.4 fold ratio) (FIG. 8). Dataset was obtained from Gene Expression Omnibus database at the National Center for Bioinformatics and analyzed for KLRG1 expression. Accordingly, T cell prolymphocytic leukemia is a particularly attractive target for therapies according to the present invention.

### Example 9: Superior Purity of Fucosylated ABC0008 Compared to HG1D03

Examples 9-12 refer to antibodies designated ABC108 and HG1D03. ABC008 is an antibody characterized by the amino acid sequences presented in Table 2, SEQ ID NO: 4-SEQ ID NO: 13, with the antibody further being afucosylated. ABC108 has identical amino acid sequences to ABC008 and retains wild-type fucosylation. HG1D03 is a humanized antibody exerting a depletion effect on KLRG1′ T cells. HG1D03 is described in more detail, including heavy and kappa chain variable region sequences, in U.S. Pat. No. 11,180,561.

ABC108 and HG1D03 were produced using the ExpiCho expression system (Thermo Fisher) at 1 L scale. Reducing capillary electrophoresis (rCE), non-reducing capillary electrophoresis (nrCE), and capillary isoelectric focusing (cIEF) were performed and the percentage of the main peak was measured in 3 independent experiments, in 4 hour, 7 day, and 28 day stress experiments (stress experiments described in Examples 11-12). The results show superior purity during CHO cell production of ABC108 compared to HG1D03 (Table 4).

TABLE 4
Purity of ABC108 compared to HG1D03

| Purity | rCE Main Peak | | nrCE Main Peak | | cIEF Main Peak | |
|---|---|---|---|---|---|---|
| Trial | ABC108 | HG1D03 | ABC108 | HG1D03 | ABC108 | HG1D03 |
| Trial 1 | 98.8% | 94.8% | 96.3% | 96.3% | 82.5% | 72.3% |
| Trial 2 | 98.2% | 97.7% | 97.9% | 97.8% | 81.0% | 72.9% |
| Trial 3 | 96.9% | 96.8% | 96.2% | 93.3% | 81.6% | 72.6% |
| Mean | 98.0% | 96.4% | 96.8% | 95.8% | 81.7% | 72.6% |

### Example 10. Superior Thermal Properties (Melting and Aggregation Temperatures) of ABC108 Compared to HG1D03

ABC108 and HG1D03 were produced using the ExpiCho expression system (Thermo Fisher) at 1 L scale. Melting temperature (Tm) was measured by full-spectrum fluorescence, and small molecule aggregate formation (Tagg 266) and large molecule aggregate formation (Tagg 473) were measured by static light scattering (SLS) using UNCLE (Unchained Labs, Inc.). Data were collected in 3 independent experiments, in 4 hour, 7 day, and 28 day stress experiments (stress experiments described in Examples 11-12). The results show superior thermal properties (higher melting points and aggregation temperatures) for ABC108 compared to HG1D03 (Table 5).

TABLE 5
Thermal properties of ABC108 compared to HG1D03.
Temperatures shown in degrees Celsius.

| Thermal | Tm1 | | Tagg 266 | | Tagg 473 | |
|---|---|---|---|---|---|---|
| Trial | ABC108 | HG1D03 | ABC108 | HG1D03 | ABC108 | HG1D03 |
| Trial 1 | 70.7 | 70.3 | 81.2 | 80.9 | 84.2 | 83.4 |
| Trial 2 | 70.6 | 70.2 | 81.6 | 81.0 | 82.9 | 82.3 |
| Trial 3 | 71.0 | 70.4 | 82.0 | 81.4 | 83.0 | 82.6 |
| Mean | 70.8 | 70.3 | 81.6 | 81.1 | 83.4 | 82.8 |

### Example 11: Superior Stability after 40° C. Stress Testing of ABC108 Compared to HG1D03

ABC108 and HG1D03 at 1 mg/mL in PBS were subjected to thermal stability stress testing by exposure to 40° C. for 4 hours, for 7 days, and for 28 days. Non-reducing capillary electrophoresis (nrCE) and capillary isoelectric focusing (cIEF) were performed and the percentage of the main peak measured. Tm1, Tagg 266, and Tagg 473 parameters were measured using the UNCLE platform. A temperature scan was performed from 25° C. to 95° C. at a 0.3° C. per minute scan rate. Tm values were calculated by Barycentric Mean Method (BCM) of analysis. The results for purity (Table 6) show similar rCE-measured purity, with nrCE-measured purity (e.g., 28 Days 85.5% vs 79.3%) and cIEF-measured purity (e.g., 7 Days 78.0% vs 41.6%) being improved for ABC108 vs HG1D03. The results for thermal properties (Table 7) show similar or slightly superior thermal properties for ABC108 vs HG1D03.

TABLE 6
Purity after 40° C. stress of ABC108 and HG1D03

| 40 C. | | rCE Main Peak | | nrCE Main Peak | | cIEF Main Peak | |
|---|---|---|---|---|---|---|---|
| TimePoint | | ABC108 | HG1D03 | ABC108 | HG1D03 | ABC108 | HG1D03 |
| 4 | Hours | 98.5% | 98.5% | 95.2% | 92.6% | 85.7% | 75.4% |
| 7 | Days | 96.0% | 97.5% | 97.1% | 98.0% | 78.0% | 41.6% |
| 28 | Days | 92.1% | 90.5% | 85.5% | 79.3% | N/A | N/A |

TABLE 7
Thermal properties after 40° C. stress of ABC108
and HG1D03. Temperatures shown in degrees Celsius

| 40 C. | | Tm1 | | Tagg 266 | | Tagg 473 | |
|---|---|---|---|---|---|---|---|
| TimePoint | | ABC108 | HG1D03 | ABC108 | HG1D03 | ABC108 | HG1D03 |
| 4 | Hours | 70.3 | 69.8 | 80.9 | 80.0 | 83.3 | 82.0 |
| 7 | Days | 67.3 | 67.4 | 78.1 | 78.1 | 79.9 | 80.0 |
| 28 | Days | 70.1 | 69.6 | 81.1 | 80.2 | 82.5 | 82.0 |

### Example 12: Superior Stability after Low pH and High pH Stress Testing of ABC108 Compared to HG1D03

ABC108 and HG1D03 at 1 mg/mL in PBS were subjected to low pH (3.5) and high pH (8.5) stress testing for 4 hours, for 7 days, and for 28 days. The results for purity (Table 8) show improved purity measured by highly sensitive capillary isoelectric focusing (cIEF) measured purity for ABC108 vs HG1D03. The results for thermal properties (Tables 9-10) show similar or slightly superior thermal properties for ABC108 vs HG1D03.

TABLE 8
Purity after pH 3.5 and pH 8.5 stress of ABC108 and HG1D03

| pH 3.5 | | cIEF Main Peak | | pH 8.5 | | cIEF Main Peak | |
|---|---|---|---|---|---|---|---|
| TimePoint | | ABC108 | HG1D03 | TimePoint | | ABC108 | HG1D03 |
| 4 | Hours | 84.0% | 72.8% | 4 | Hours | 78.8% | 74.0% |
| 7 | Days | 81.4% | 73.2% | 7 | Days | 64.5% | 57.8% |
| 28 | Days | 79.6% | 66.3% | 28 | Days | 72.8% | 66.5% |

TABLE 9
Thermal properties after pH 3.5 stress of ABC108
and HG1D03. Temperatures shown in degrees Celsius

| pH 3.5 | | Tm1 | | Tagg 266 | | Tagg 473 | |
|---|---|---|---|---|---|---|---|
| TimePoint | | ABC108 | HG1D03 | ABC108 | HG1D03 | ABC108 | HG1D03 |
| 4 | Hours | 70.5 | 69.9 | 80.7 | 80.3 | 82.6 | 82.0 |
| 7 | Days | 70.3 | 69.9 | 80.4 | 79.6 | 82.8 | 82.2 |
| 28 | Days | 70.4 | 69.8 | 80.6 | 80.1 | 82.5 | 82.0 |

TABLE 10
Thermal properties after pH 8.5 stress of ABC108
and HG1D03 Temperatures shown in degrees Celsius

| pH 8.5 | | Tm1 | | Tagg 266 | | Tagg 473 | |
|---|---|---|---|---|---|---|---|
| TimePoint | | ABC108 | HG1D03 | ABC108 | HG1D03 | ABC108 | HG1D03 |
| 4 | Hours | 70.4 | 70.0 | 80.8 | 80.2 | 82.6 | 81.9 |
| 7 | Days | 70.2 | 69.9 | 80.0 | 79.6 | 82.4 | 82.1 |
| 28 | Days | 70.4 | 70.3 | 80.4 | 80.4 | 83.3 | 82.9 |

### Example 13: Depletion of KLRG1+ Blood Cells by ABC008 in Cynomolgus Monkeys

Cynomolgus monkeys were administered vehicle control or ABC008 at 5 mg/kg, 25 mg/kg, or 50 mg/kg subcutaneously. Blood immune cell populations were monitored by FACS. ABC008 resulted in near complete depletion of the KLRG1+CD8+ T cell population (FIG. 9).

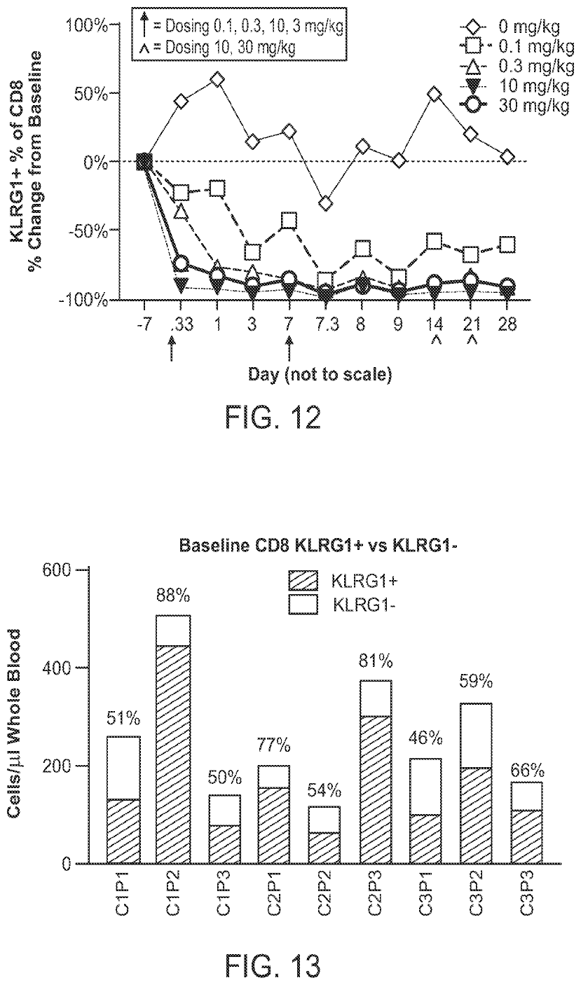

In another experiment, Cynomolgus monkeys were administered vehicle control or ABC008 at 0.1 mg/kg, 0.3 mg/kg, 10 mg/kg, or 30 mg/kg subcutaneously, with multiple doses according to the regimens indicated in FIG. 12. Blood immune cell populations were monitored by FACS. ABC008 at 0.1 mg/kg resulted in greater than approximately 50% reduction in the KLRG1+CD8+ T cell population. Depletion of the KLRG1+CD8+ T cell population was nearly complete when 0.3 mg/kg or more ABC008 was administered (FIG. 12).

### Example 14: Depletion of KLRG1+ Blood Cells by ABC0008 in Patients with IBM

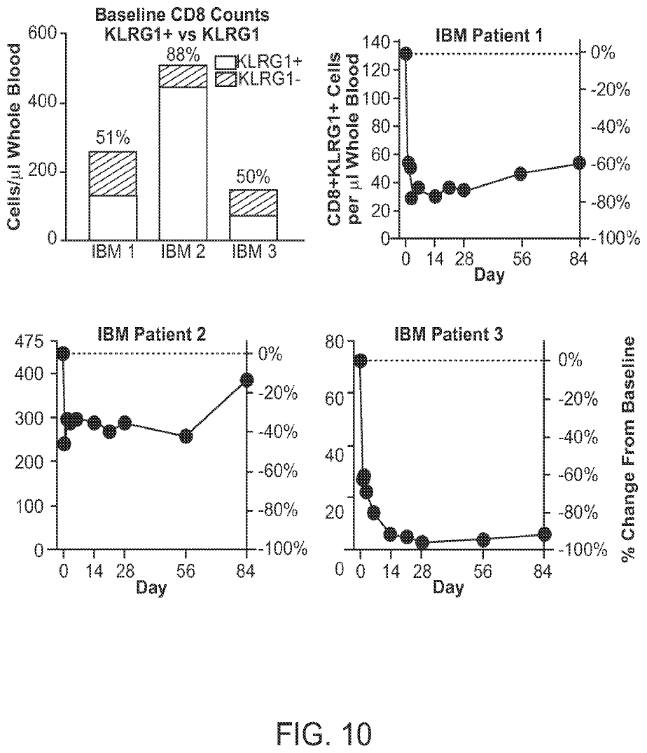

Three patients with IBM were administered single doses of ABC008 (0.1 mg/kg subcutaneously) in clinical trial NCT04659031. Blood immune cell populations were monitored by FACS. At baseline, KLRG1+% of CD8 T cells was 50-88%. ABC0008 resulted in peak depletion of 46-96% of the KLRG1+CD8+ T cell population (FIG. 10).

The first three patients were considered a first cohort. Two additional cohorts, each of three patients with IBM, were administered single doses of ABC0008 subcutaneously in clinical trial NCT04659031. The doses were: cohort 1: 0.1 mg/kg; cohort 2: 0.5 mg/kg; cohort 3: 2.0 mg/kg.

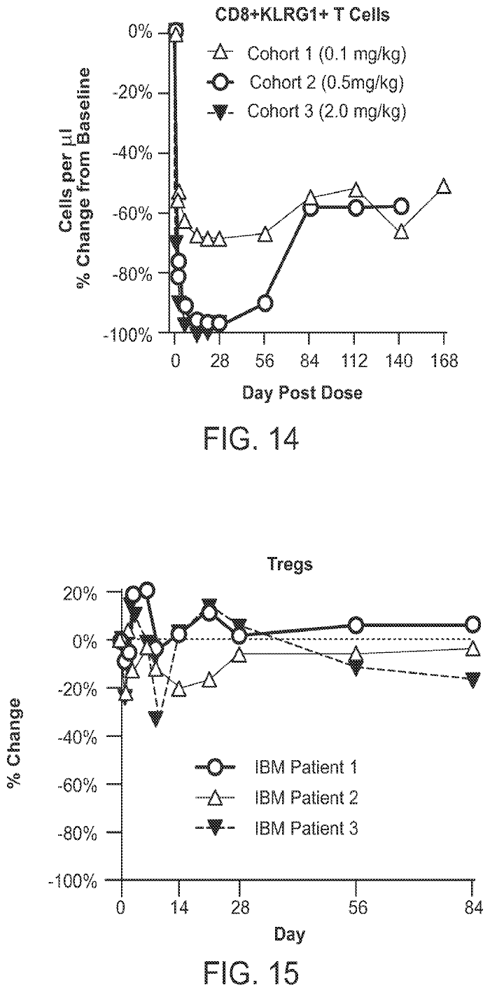

Blood immune cell populations were monitored by FACS. As shown in FIG. 13, at baseline, the percentage of KLRG1+CD8 T cells ranged from 46-88%. As shown in FIG. 14, administration of ABC0008 resulted in peak depletion of about 70% of the KLRG1+CD8+ T cell population in cohort 1, and about 95% in both cohorts 2 and 3.

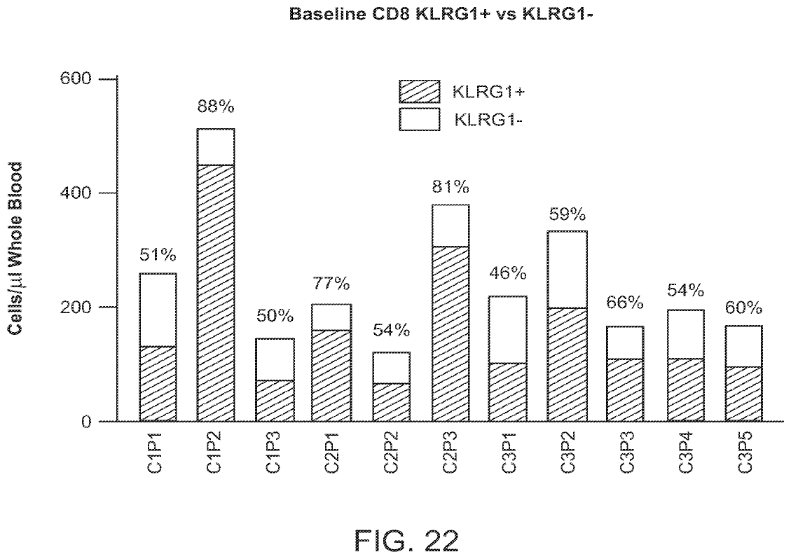

Two additional patients met the requirement for inclusion in cohort 3 and anonymized as C3P4 and C3P5. As shown in FIG. 22, at baseline, the percentage of KLRG1+CD8 T cells in these two patients ranged from 54-60%.

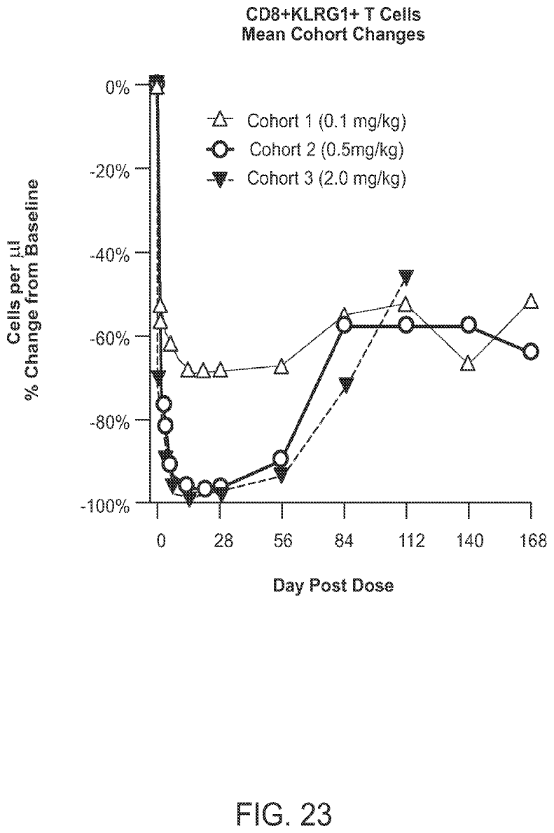

FIG. 23 supplements FIG. 14 by following cohort 3 for 112 days. As shown in FIG. 23, across all three cohorts, the KLRG1+CD8+ T cell population for cohorts 1 and 2 remained about 50-70% below baseline from days 84 to 168 post dose. The KLRG1+CD8+ T cell population for cohort 3 was about 40% depletion 112 days post dose.

### Example 15. Depletion of Large Granular Lymphocytes (LGLs) by ABC0008 in Patients with IBM

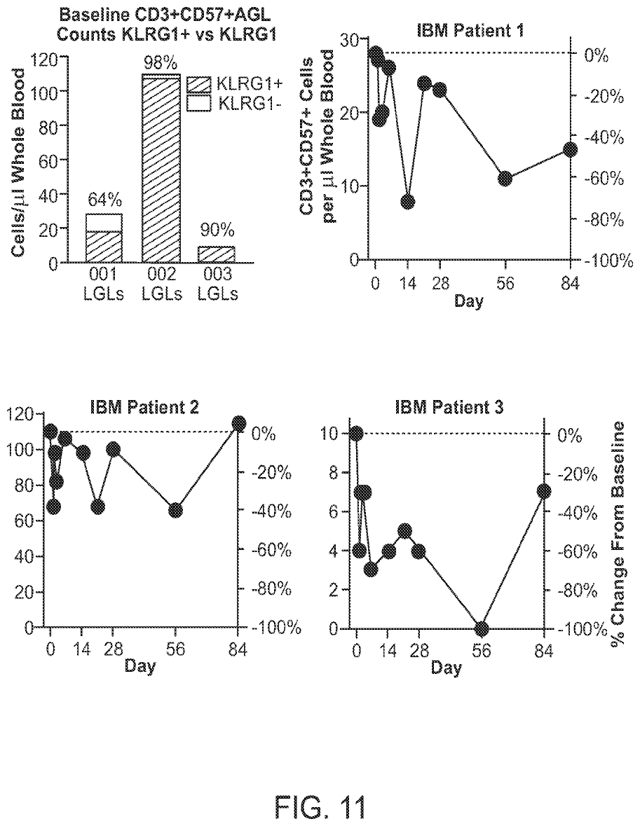

Three patients with IBM were administered single doses of ABC0008 0.1 mg/kg subcutaneously in clinical trial NCT04659031. Blood immune cell populations were monitored by FACS. At baseline, KLRG1+% of large granular lymphocytes (CD3+CD57+ LGLs) T cells was 64-98%. Administration of ABC0008 resulted in peak depletion of 40-100% of the CD3+CD57+ LGL T cell population (FIG. 11).

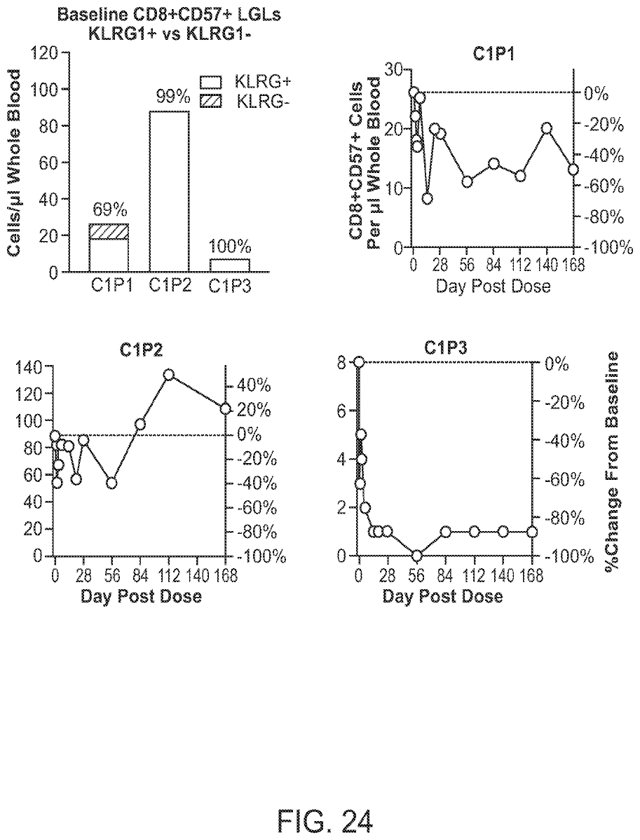

The patients were followed for 168 days. Two showed sustained depletion of the CD3+CD57+ LGL T cell population (FIG. 24).

Two additional cohorts, one of three patients and one of five patients, were administered single doses of ABC0008 as described for cohorts 2 and 3 in Example 14, and blood immune cell populations were monitored by FACS for 168 days (cohort 2) or 28-112 days (cohort 3).

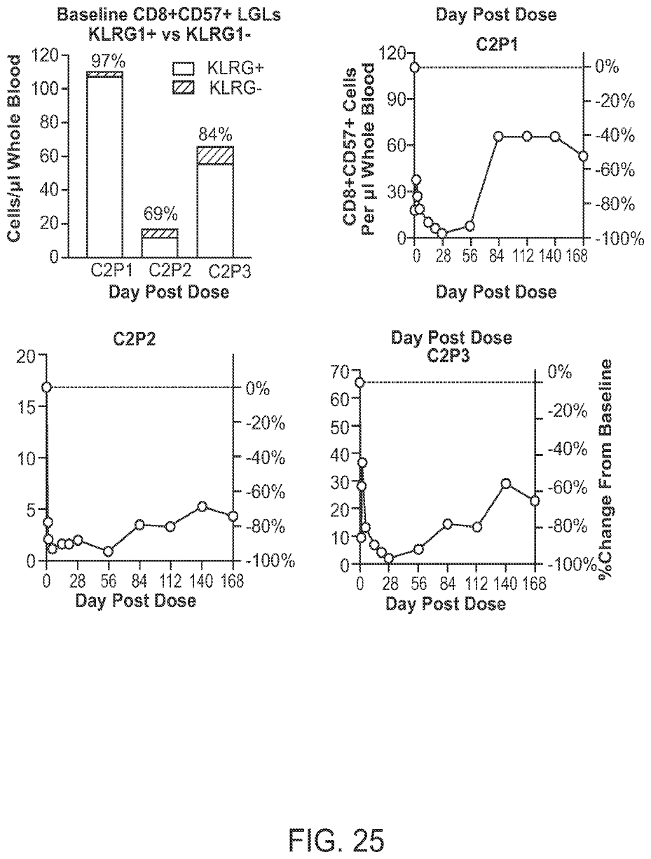

For cohort 2, baseline KLRG1+% of large granular lymphocytes (CD3+CD57+ LGLs) T cells was 69-97%. Administration of ABC0008 resulted in peak depletion of about 90-100% of the CD3+CD57+ LGL T cell population, and sustained depletion of about 40-80% for the monitoring period (FIG. 25).

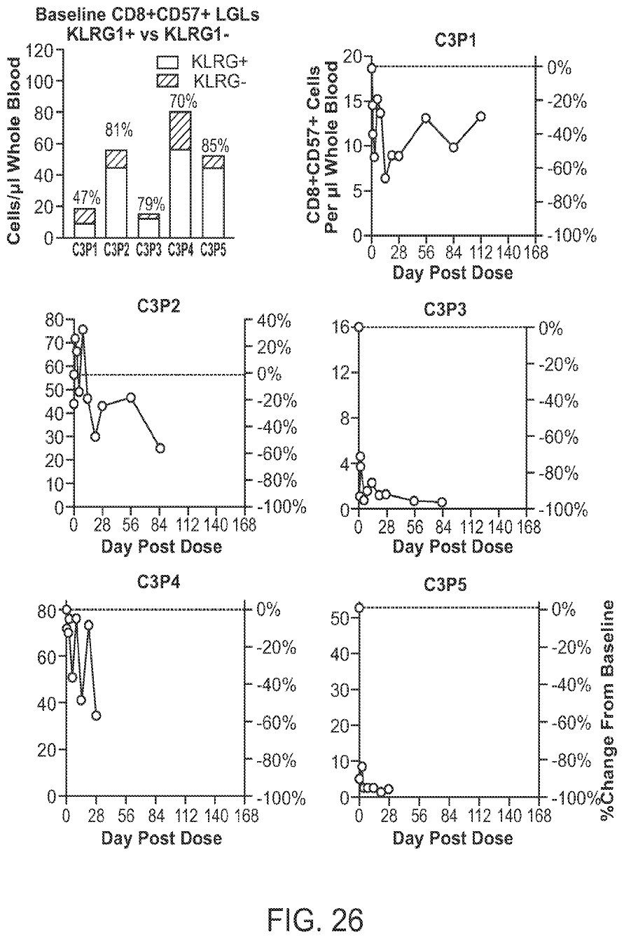

For cohort 3, baseline KLRG1+% of large granular lymphocytes (CD3+CD57+ LGLs) T cells was 47-85%. Administration of ABC0008 resulted in peak depletion of about 50-100% of the CD3+CD57+ LGL T cell population, and sustained depletion of about 25-55% for patient 1 (monitored for 112 days) and over 90% for patient 3 (monitored for 84 days) (FIG. 26).

### Example 16. Depletion of Large Granular Lymphocytes (LGLs) by ABC0008 in Patients with T Cell Large Granular Lymphocytic Leukemia (T-LGLL)

Three patients with T cell large granular lymphocytic leukemia (T-LGLL) are administered ABC0008 0.25 mg/kg subcutaneously at Day 1 and at Weeks 1, 12, 24, and 36 in a clinical trial. Blood immune cell populations are monitored by FACS. The group receiving ABC008 shows depletion of LGL cells.

### Example 17: Regulatory T Cells (Tregs) are Spared by ABC0008 in Patients with IBM

Three patients with IBM were subcutaneously administered single doses of ABC008 (0.1 mg/kg) in clinical trial NCT04659031. Blood immune cell populations were monitored by FACS. ABC008 resulted in negligible depletion (<˜20% from 21 to 84 days after administration) of Treg cell populations (FIG. 15).

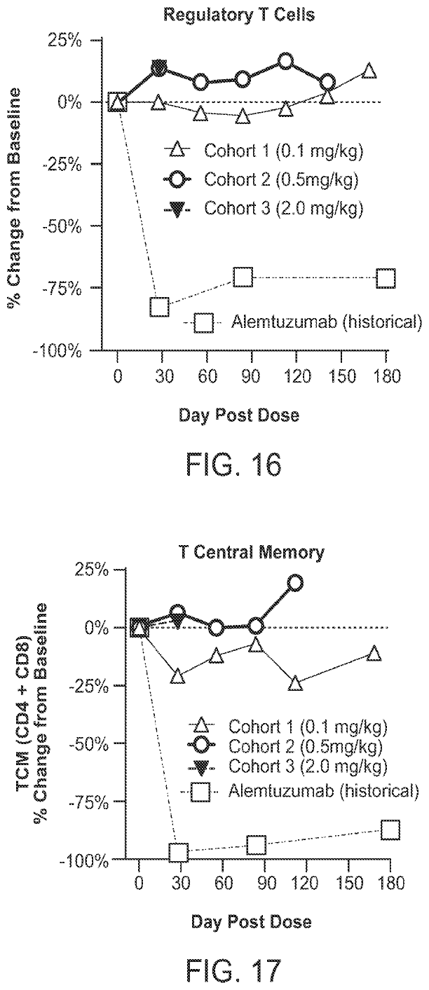

Three cohorts, each including three patients with IBM, were subcutaneously administered ABC008 as set forth in Example 14. Treg cell populations were monitored by FACS and compared with historical data from trials of alemtuzumab against multiple sclerosis. Administration of ABC008 resulted in negligible depletion (peak depletion <˜10%) of Tregs over 180 days (FIG. 16). In contrast, alemtuzumab depleted ˜75% of Tregs over the same period (FIG. 16).

### Example 18: Central Memory T Cells are Spared by ABC0008 in Patients with IBM

Three cohorts, each including three patients with IBM, were subcutaneously administered ABC008 as set forth in Example 14. Central memory T cell populations were monitored by FACS and compared with historical data from trials of alemtuzumab against multiple sclerosis. Administration of ABC008 resulted in negligible depletion (peak depletion <˜25%) of central memory T cells over 180 days (FIG. 17). In contrast, alemtuzumab depleted >˜80% of central memory T cells over the same period (FIG. 17).

Three cohorts, two including three patients with IBM and the third including five patients, were subcutaneously administered ABC0008 as set forth in Example 14. Central memory T cell populations were monitored by FACS and compared with historical data from trials of alemtuzumab against multiple sclerosis. Administration of ABC0008 resulted in less than about 60% peak depletion of central memory T cells over 180 days (FIG. 28). In contrast, alemtuzumab depleted >˜80% of central memory T cells over the same period (FIG. 28).

### Example 19: Pharmacokinetics of ABC008 in Patients with IBM

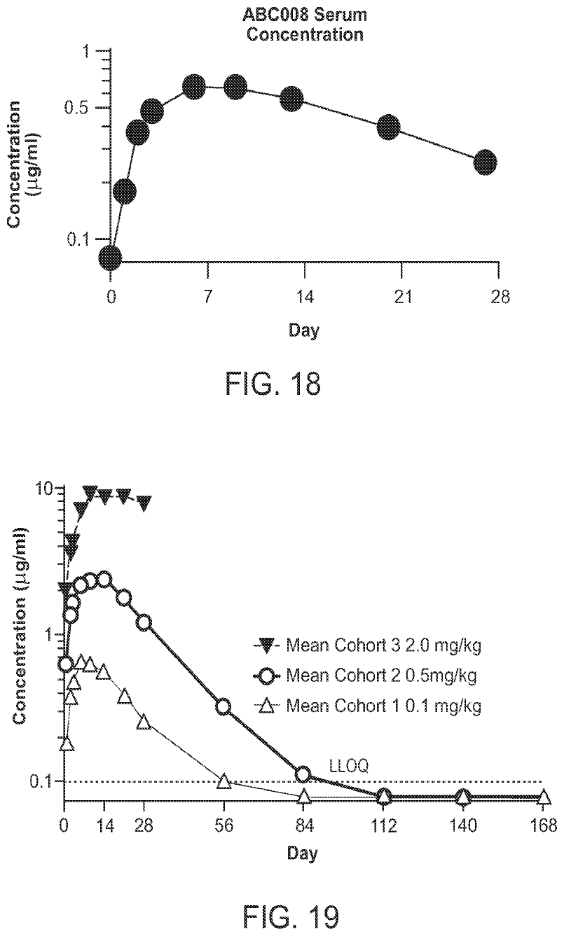

A patient with IBM was subcutaneously administered a single dose of ABC0008 (0.1 mg/kg) in clinical trial NCT04659031. As shown in FIG. 18, ABC0008 displayed a long absorption and slow clearance, typical of monoclonal antibody therapies.

Three cohorts, each including three patients with IBM, were subcutaneously administered ABC0008 as set forth in Example 14. As shown in FIG. 19, ABC0008 displayed a long absorption and slow clearance, typical of monoclonal antibody therapies.

### Example 20: Impact of ABC008 on Disease Severity in Patients with IBM

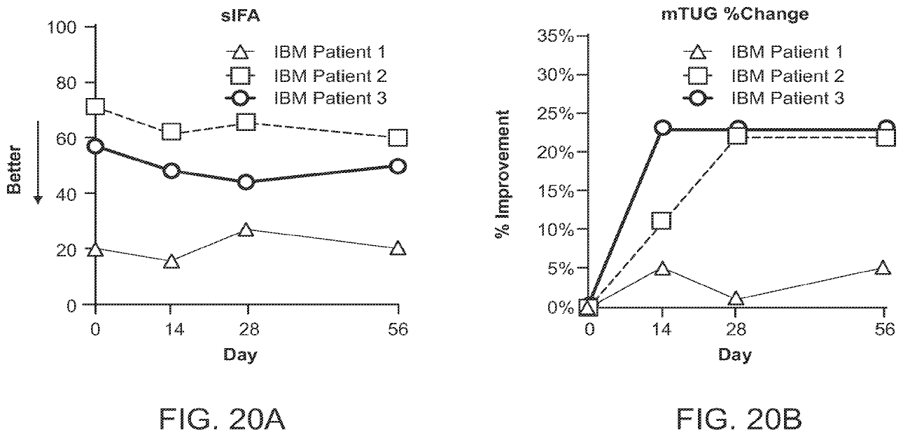

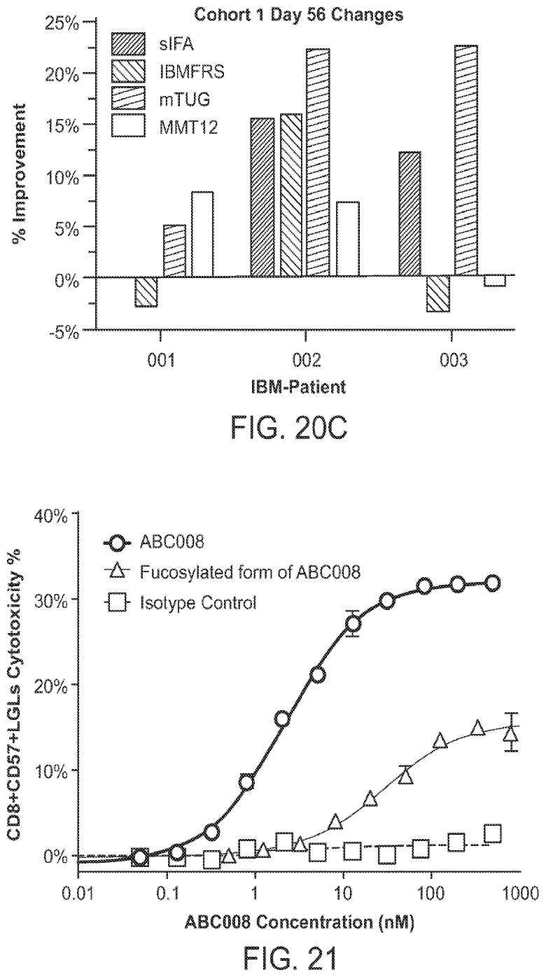

The disease severity in three patients with IBM was evaluated by sporadic Inclusion Body Myositis Physical Functioning Assessment (sIFA), Inclusion Body Myositis Functional Rating Scale (IBMFRS), modified Timed Up and Go (mTUG), and Manual Muscle Testing (MMT12) prior to subcutaneously administering single doses of ABC0008 (0.1 mg/kg) in clinical trial NCT04659031, followed by evaluation using the same assessments over 56 days. FIG. 20A shows absolute sIFA score at various time points after ABC0008 administration. FIG. 20B shows percentage improvement in mTUG at various time points. FIG. 20C summarizes changes in all four assessments after 56 days. The most pronounced improvements were seen in patient #2, who had the highest IBM disease severity at baseline.

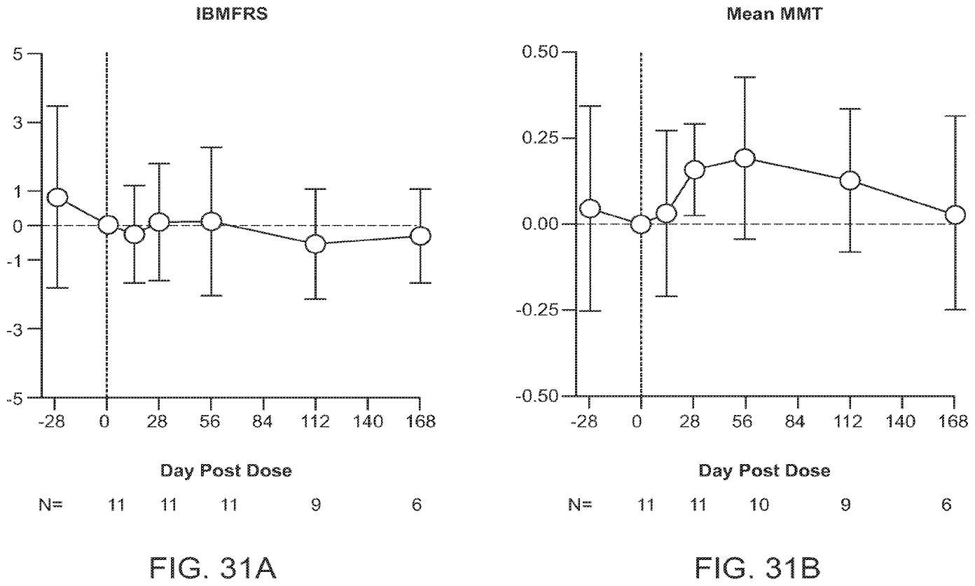

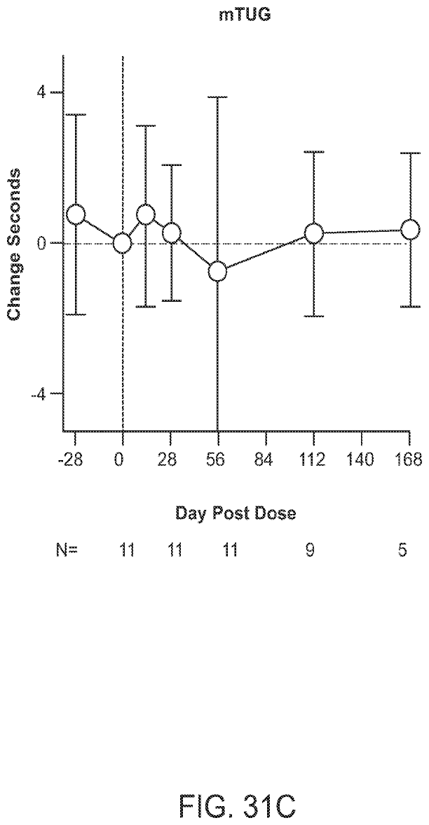

FIG. 31A-31C show trends toward functional stability or improvement in IBMFRS, MMT, and mTUG until at least day 56 for combined functional readouts across three cohorts of IBM patients receiving single ascending subcutaneous doses of ABC0008 (0.1, 0.5 and 2.0 mg/kg) in the same clinical trial.

### Example 21: In Vitro Depletion of Human CD8+CD57+ LGLs by ABC0008 or ABC108

Purified human CD8+CD57+ LGLs were incubated with ABC008, ABC108 (the fucosylated form of ABC008), or isotype control in concentrations from about 0.1 nM to about 1 mM. ABC008 showed greater potency than ABC108 against CD8+CD57+ LGLs (FIG. 21).

### Example 22: Naive T Cells are Spared by ABC0008 in Patients with IBM

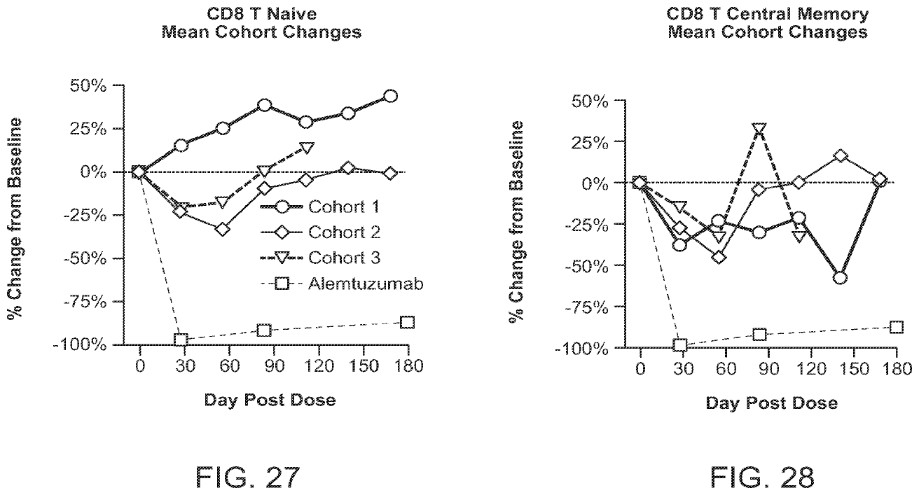

Three cohorts, two including three patients with IBM and the third including five patients, were subcutaneously administered ABC008 as set forth in Example 14. Naive T cell populations were monitored by FACS and compared with historical data from trials of alemtuzumab against multiple sclerosis. Administration of ABC008 resulted in less than about 40% peak depletion of naïve T cells over 180 days (FIG. 27). In contrast, alemtuzumab depleted >˜80% of naïve T cells over the same period (FIG. 27).

### Example 23: CD8 Effector Memory T Cells are Depleted by ABC008 in Patients with IBM

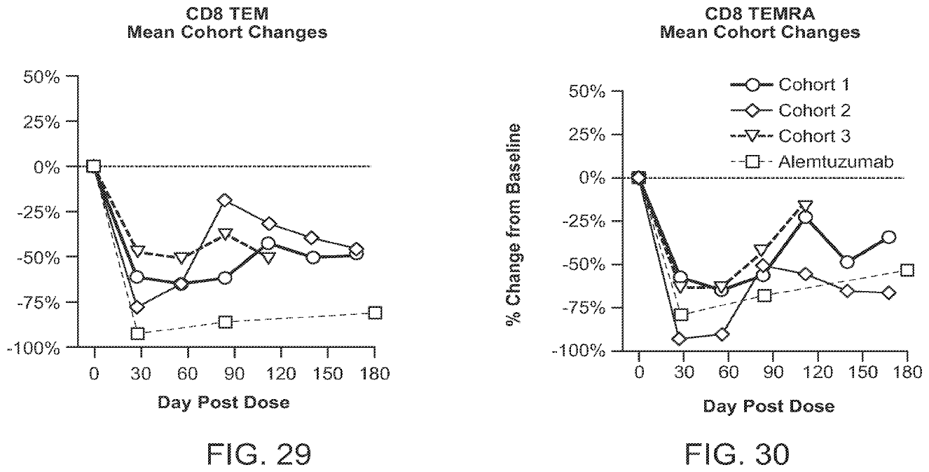

Three cohorts, two including three patients with IBM and the third including five patients, were subcutaneously administered ABC008 as set forth in Example 14. CD8 effector memory T (TEM) cell populations were monitored by FACS and compared with historical data from trials of alemtuzumab against multiple sclerosis. Administration of ABC008 resulted in about 50-75% peak depletion of TEM cells with sustained depletion of about 30-50% over 112-180 days (FIG. 29). In comparison, alemtuzumab depleted >˜80% of TEM cells over the same period (FIG. 29).

### Example 24: CD8 Terminally Differentiated Effector Memory T Cells are Depleted by ABC008 in Patients with IBM

Three cohorts, two including three patients with IBM and the third including five patients, were subcutaneously administered ABC008 as set forth in Example 14. CD8 terminally differentiated effector memory T (TEMRA) cell populations were monitored by FACS and compared with historical data from trials of alemtuzumab against multiple sclerosis. Administration of ABC008 resulted in about 55-95% peak depletion of TEMRA cells with sustained depletion of about 20-70% over 112-180 days (FIG. 30). In comparison, alemtuzumab depleted >˜80% of TEMRA cells over the same period (FIG. 29).

One of ordinary skill in the art will recognize the numerous modifications and variations that may be performed without altering the spirit or scope of the present disclosure. Such modifications and variations are encompassed within the scope of the present disclosure. The entire contents of all references, patents, and published patent applications cited throughout this application are herein incorporated by reference.

### REFERENCES

Swerdlow S H, et al. The 2016 revision of the World Health Organization classification of lymphoid neoplasms. Blood 2016; 127(20): 2375-90.
Takata K, et al. Primary cutaneous NK/T-cell lymphoma, nasal type and CD56-positive peripheral T-cell lymphoma: a cellular lineage and clinicopathologic study of 60 patients from Asia. The American journal of surgical pathology 2015; 39(1): 1-12.
Zing NPC, et al. Peripheral T-Cell Lymphomas: Incorporating New Developments in Diagnostics, Prognostication, and Treatment Into Clinical Practice-PART 2: ENKTL, EATL, Indolent T-Cell LDP of the GI Tract, ATLL, and Hepatosplenic T-Cell Lymphoma. Oncology (Williston Park) 2018; 32 (8): e83-e9.

1. An antibody, or a fragment thereof, that specifically binds to an extracellular domain of KLRG1 comprising a heavy chain variable region comprising an amino acid sequence of SEQ ID NO:4, and a light chain variable region comprising light chain complementarity determining regions (CDRs) comprising amino acid sequences SEQ ID NO:11 (CDR-L1), SEQ ID NO:12 (CDR-L2), and SEQ ID NO:13 (CDR-L3).

2. The antibody, or fragment thereof, of claim 1, wherein the light chain variable region comprises an amino acid sequence of SEQ ID NO:5.

3. The antibody, or fragment thereof, of claim 1, wherein the antibody, or fragment thereof, comprises a heavy chain comprising an amino acid sequence of SEQ ID NO:6.

4. The antibody, or fragment thereof, of claim 1, wherein the antibody, or fragment thereof, comprises a light chain comprising an amino acid sequence of SEQ ID NO:7.

5. The antibody, or fragment thereof, of claim 1, wherein the antibody, or fragment thereof, comprises a heavy chain comprising an amino acid sequence of SEQ ID NO:6 and a light chain comprising an amino acid sequence of SEQ ID NO:7.

6. The antibody, or fragment thereof, of claim 1, wherein the antibody, or fragment thereof, specifically binds the epitope PLNFSRI (SEQ ID NO:14), or a fragment thereof comprising at least five contiguous amino acids.

7. The antibody, or fragment thereof, of claim 1, wherein the antibody, or fragment thereof, comprises a monoclonal antibody, or a fragment or derivative thereof.

8. The antibody, or fragment thereof, of claim 1, wherein the antibody, or fragment thereof, comprises a humanized antibody, or a fragment thereof.

9. The antibody, or fragment thereof, of claim 1, wherein the KLRG1 comprises human KLRG1 or cynomolgus KLRG1.

10. A method of depleting KLRG1-expressing T cells and/or NK cells in a subject in need thereof comprising:
delivering to said subject a therapeutically effective amount of an antibody, or fragment thereof, of claim 1, thereby depleting KLRG1-expressing T cells and/or NK cells in the subject.

11. A method of treating a disorder associated with excess KLRG1-expressing T cells in a subject in need thereof, comprising:
delivering to said subject a therapeutically effective amount of an antibody, or fragment thereof, of claim 1, thereby reducing the excess KLRG1-expressing T-cells in the subject.

12. The method of claim 11, wherein the disorder is a transplant disorder.

13. The method of claim 11, wherein the disorder is an autoimmune disease.

14. The method of claim 11, wherein the disorder is inclusion body myositis (IBM).

15. A method of treating cancer in a subject, wherein the cancer comprises cancer cells that express KLRG1, comprising:
delivering to said subject a therapeutically effective amount of an antibody, or fragment thereof, of claim 1, wherein the delivery to the subject depletes the cancer cells expressing KLRG1.

16. An adjunct therapy for treatment of cancer in a subject, wherein the subject is undergoing checkpoint therapy, regardless of whether said cancer expresses KLRG1, the adjunct therapy comprising:
delivering to said subject a therapeutically effective amount of an antibody, or fragment thereof, of claim 1, wherein delivery depletes KLRG1-expressing pathogenic T cells and/or NK cells attacking self-tissues in the subject.

17. A method of depleting KLRG1-expressing cells in a mixed population of cells, wherein said KLRG1-expressing cells comprise one or more cells selected from the group consisting of T cells, NK cells, cancer cells, and combinations thereof, comprising:
delivering to said mixed population of cells a therapeutically effective amount of an antibody, or fragment thereof, of claim 1, wherein the delivery depletes KLRG1-expressing T cells, NK cells, cancer cells, and combinations thereof, in the mixed population of cells.

18. A method of selectively depleting KLRG1-expressing CD8 effector T cells with relative sparing of naïve T cells and/or regulatory T cells, comprising:
delivering to a subject a therapeutically effective amount of an antibody, or fragment thereof, of claim 1, thereby selectively depleting KLRG1-expressing CD8 effector T cells.

19. A pharmaceutical composition, comprising at least one antibody, or fragment thereof, of claim 1 and a pharmaceutically acceptable carrier.

20. A kit, comprising at least one antibody, or fragment thereof, of claim 1 and instructions for use.


SEQUENCE LISTING (16 sequences)

SEQ ID NO: 1 - protein, 130 aa, Homo sapiens
LCQGSNYSTCASCPSCPDRWMKYGNHCYYFSVEEKDWNSSLEFCLARDSHLLVITDNQEMSLLQVFLSEAFCWIGLRNNSGWRWEDGSPLNFSRISSNSFVQTCGAINKNGLQASSCEVPLHWVCKKVRL

SEQ ID NO: 2 - protein, 130 aa, Macaca fascicularis
LCQGSKYSTCASCPSCPDHWMKYGNHCYYFSVEKKDWISSLEFCLARDSHLLMITDKQEMSLLQDFLSEAFHWVGLRNNSGWRWEDGSPLNFSRIYSNSLVQTCGAINKNSLQASSCEVSLQWVCKKVSP

SEQ ID NO: 3 - protein, 728 aa, Homo sapiens
DWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 4 - protein, 119 aa, synthetic construct
QVQLVQSGAEVKKPGASVKVSCKASGYTFTDHNMHWVKQATGQGLEWFGFINPNTGVTRYNQKFQGRVTLTINKAISTAYLELSSLRSEDTAVYYCARDYYGSAWFAYWGQGTLVTVSS

SEQ ID NO: 5 - protein, 112 aa, synthetic construct
DIVMTQSPDSLAVSLGERATINCKSSQTLLYSSDQKNYLAWYQQKPGQPPKLLIYWASTRESGVPDRFSGSGSGTDFTLTISSLQAEDVAVYYCQQYYNYPTFGGGTKVEIK

SEQ ID NO: 6 - protein, 449 aa, synthetic construct
QVQLVQSGAEVKKPGASVKVSCKASGYTFTDHNMHWVKQATGQGLEWFGFINPNTGVTRYNQKFQGRVTLTINKAISTAYLELSSLRSEDTAVYYCARDYYGSAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 219 aa, synthetic construct
DIVMTQSPDSLAVSLGERATINCKSSQTLLYSSDQKNYLAWYQQKPGQPPKLLIYWASTRESGVPDRFSGSGSGTDFTLTISSLQAEDVAVYYCQQYYNYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 7 aa, synthetic construct
GYTFTDH

SEQ ID NO: 9 - protein, 6 aa, synthetic construct
NPNTGV

SEQ ID NO: 10 - protein, 10 aa, synthetic construct
DYYGSAWFAY

SEQ ID NO: 11 - protein, 17 aa, synthetic construct
KSSQTLLYSSDQKNYLA

SEQ ID NO: 12 - protein, 7 aa, synthetic construct
WASTRES

SEQ ID NO: 13 - protein, 8 aa, synthetic construct
QQYYNYPT

SEQ ID NO: 14 - protein, 7 aa, Homo sapiens
  source 1..7: note = also found in Macaca fascicularis
PLNFSRI

SEQ ID NO: 15 - protein, 15 aa, synthetic construct
GGGGSGGGGSGGGGS

SEQ ID NO: 16 - protein, 5 aa, synthetic construct
CNGRC